(19) United States
(12) Patent Application Publication
(10) Pub. No.: US 2015/0355187 A1
(43) Pub. Date: Dec. 10, 2015

(54) MIXED PAYLOAD CONTAINING POLYMERS AND USE THEREOF

(71) Applicant: AKRIVIS TECHNOLOGIES, LLC, Cambridge, MA (US)

(72) Inventors: Joel Berniac, Stoneham, MA (US); Rajiv Panwar, Wilmington, MA (US)

(21) Appl. No.: 14/654,228

(22) PCT Filed: Dec. 19, 2013
(86) PCT No.: PCT/US2013/076469

§ 371 Date: Jun. 19, 2015

Related U.S. Application Data
(60) Provisional application No. 61/740,142, filed on Dec. 20, 2012.

(51) Int. Cl. G01N 33/58; A61K 47/48; C12N 9/08; C12Q 1/68

(52) U.S. Cl. CPC G01N 33/581; C12Q 1/6841; G01N 33/582; A61K 47/48215; A61K 47/48315; A61K 47/48723; C12N 9/0065; C12Y 111/01007

(57) ABSTRACT

The invention provides methods for delivery of payloads to targets in samples using mixed payload polymers. The invention further provides reagents and kits for practicing the methods of the invention. The invention also provides methods for preparation of reagents for use in the methods of the invention.

### CROSS-REFERENCE TO RELATED APPLICATION

This application claims priority to U.S. Provisional Patent Application No. 61/740,142, filing date Dec. 20, 2012. The application is incorporated herein by reference in its entirety.

### BACKGROUND

Antibodies, and antigen binding fragments thereof, are useful for the delivery of specific payloads (e.g., therapeutic or imaging agents, detectable labels) to target antigens. The payload can be a detectable label in vitro for detection and/or quantitation of the antigen, e.g., for diagnostic assays such as ELISA, immunofluorescence or other immunoassays. An immunoassay uses antibodies to detect a compound of interest. However, the sensitivity of this detection is generally limited to the amount of detectable label that can be carried either on an antibody, for a direct binding assay, or on a secondary detection reagent (e.g., an anti-immunoglobulin antibody often referred to as a secondary antibody). For example, in existing immunoassays, if too many detectable labels, e.g., horse radish peroxidase (HRP) or alkaline phosphatase, are attached to the primary or secondary antibody, the binding of the antibody to the antigen can be inhibited by steric hindrance or denaturation of the labeled antibody. Therefore, in order to obtain more signal, additional antibody or probe must be added, increasing background. This, in turn, reduces the sensitivity of the assay, limiting the capability of the assay to detect minute quantities of the target antigen.

Alternatively, an antibody can also be used to deliver a payload in vivo e.g., imaging agents for detection by magnetic resonance imaging (MRI), computerized axial tomography (CAT scan), or computed tomography (CT scan); or therapeutic agents, e.g., radiopharmaceuticals or drug molecules, to a target site, e.g., a tumor. By increasing the proportion of the therapeutic agent delivered to the target site, the therapeutic index of some agents can be increased by reducing the needed dose to observe a beneficial effect. However, as with in vitro detection reagents, the number of payload molecules that can be attached to a single antibody is limited as binding of the antibody to its target antigen can be limited by the presence of the payload.

### SUMMARY OF THE INVENTION

The invention provides mixed payload polymers for delivery of payload molecules to a target site. The mixed payload polymers and methods provided herein for their use result in an increased delivery of payload to the target site. The methods can be used, for example, for diagnostic and therapeutic methods, as well as for laboratory research methods. The invention also provides kits including mixed payload polymers for practicing the methods of the invention.

The invention provides methods of delivering a payload in a mixed payload polymer to a target molecule, the method comprising, contacting the target molecule with a bispecific ligand comprising a first binding site and a second binding site wherein the first binding site binds specifically to a payload that is not the smallest payload on the polymer and the second binding site binds specifically to the target which is not a payload; contacting the bispecific ligand with a mixed payload polymer comprising a tether molecule linked to two or more distinct payload molecules (e.g., 2, 3, 4, 5, etc.), preferably three or more distinct payload molecules; wherein the payload is delivered to the target molecule, the detection sensitivity of a molecule for detection is 10⁻¹⁵ g/ml or less, 10⁻¹⁶ g/ml or less, 10⁻¹⁷ g/ml or less, 10⁻¹⁸ g/ml or less, 10⁻¹⁹ g/ml or less, 10⁻²⁰ g/ml or less, or 10⁻²¹ g/ml or less, and the molecule for detection is the target or is bound by the target.

The invention provides methods of delivering a payload in a mixed payload polymer to a target molecule, the method comprising, contacting the target molecule with a bispecific ligand comprising a first binding site and a second binding site wherein the first binding site binds specifically to a payload that is not the smallest payload on the polymer and the second binding site binds specifically to the target which is not a payload; contacting the bispecific ligand with a mixed payload polymer comprising a tether molecule linked to two or more distinct payload molecules, preferably three or more distinct payload molecules, wherein at least one of the distinct payload molecules attached to the tether have at least 50%, at least 55%, at least 60%, at least 65%, at least 70%, at least 75%, at least 80%, at least 85%, at least 90%, or at least 95% of the activity of a molar equivalent of payload molecules not attached to a tether; whereby the payload is delivered to the target molecule.

The invention provides methods of delivering a payload in a mixed payload polymer to a target molecule, the method comprising, contacting the target molecule with a bispecific ligand comprising a first binding site that is not the smallest payload on the polymer and a second binding site wherein the first binding site binds specifically to a payload and the second binding site binds specifically to the target which is not a payload; contacting the bispecific ligand with a mixed payload polymer comprising a tether molecule linked to two or more distinct payload molecules, preferably three or more distinct payload molecules, wherein the payload molecule captured by the bispecific ligand has a molecular weight of at least 2 kDa, 3 kDa, 5 kDa, 10 kDa, 15 kDa, 20 kDa, 25 kDa, 30 kDa, 35 kDa, 40 kDa, 50 kDa, 60 kDa, 70 kDa, 80 kDa, 90 kDa, 100 kDa, 110 kDa, 120 kDa, 130 kDa, 140 kDa, 150 kDa, 160 kDa, 170 kDa, 180 kDa, 190 kDa, 200 kDa, 250 kDa, 300 kDa, 350 kDa, 400 kDa, 450 kDa, 500 kDa, 600 kDa, 700 kDa, 800 kDa, 900 kDa, 1000 kDa, or more, or any range bracketed by any of the values listed.

The invention provides methods of delivering a payload in a mixed payload polymer to a target molecule, the method comprising, contacting the target molecule with a bispecific ligand comprising a first binding site and a second binding site wherein the first binding site binds specifically to a payload that is not the smallest payload on the polymer and the second binding site binds specifically to the target which is not the payload; contacting the bispecific ligand with a mixed payload polymer; whereby the payload is delivered to the target molecule, wherein at least 0.5-fold, 1 fold, 2-fold 3-fold, 4-fold, 5-fold, 7-fold, 10-fold, 12-fold, 15-fold, 20-fold, or more payload is delivered to the target site using a bispecific ligand that binds directly to a payload that is not the smallest payload as compared to a bispecific ligand that binds the mixed payload polymer at the smallest payload moiety having a molecular weight of 10 kDa, 7 kDa, 5 kDa, 3 kDa, 2 kDa, 1 kDa, 750 Da, 500 Da or less. In certain embodiments, the captured payload has a molecular weight at least 2-fold 3-fold, 4-fold, 5-fold, 7-fold, 10-fold, 12-fold, 15-fold, 20-fold, 25-fold 30-fold, 40-fold, 50-fold, 75-fold, 100-fold, 125-fold, 150-fold, 200-fold, or more than the smallest payload.

The invention provides methods of delivering a payload in a mixed payload polymer to a nucleic acid target molecule, the method comprising, contacting the target molecule with a bispecific ligand comprising a first binding site and a second binding site wherein the first binding site binds specifically to a payload that is not the smallest payload on the polymer and the second binding site binds specifically to the nucleic acid target molecule which is not the payload; contacting the bispecific ligand with a mixed payload polymer; whereby the payload is delivered to the nucleic acid target molecule.

In certain embodiments, the methods further comprise detecting the target nucleic acid. In certain embodiments, the nucleic acid is detected without amplification.

In certain embodiments, the number of total payload molecules combined (i.e., all distinct types combined) per tether is about 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 325, 350, 375, 400, 425, 450, 475, 500, or more; or any range bracketed by any of the values listed.

In certain embodiments, the number of each individual type of payload molecules per tether is independently selected from about 1, 1.5, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 325, 350, 375, 400, 425, 450, 475, 500, or more; or any range bracketed by any of the values listed.

In certain embodiments, at least two payload molecules are linked to a single tether. In certain embodiments, at least three payload molecules are linked to a single tether. In certain embodiments, the tether is unbranched. In certain embodiments, the tether is negatively charged. In certain embodiments, the tether is a liposome, micelle, or other phospholipid complex. In certain embodiments, the tether is not a liposome, micelle, or other phospholipid complex.

In certain embodiments, each payload molecule has a molecular weight of at least about 2 kDa, 3 kDa, preferably at least 5 kDa, 10 kDa, 15 kDa, 20 kDa, 25 kDa, 30 kDa, 35 kDa, 40 kDa, 50 kDa, 60 kDa, 70 kDa, 80 kDa, 90 kDa, 100 kDa, 110 kDa, 120 kDa, 130 kDa, 140 kDa, 150 kDa, 160 kDa, 170 kDa, 180 kDa, 190 kDa, 200 kDa, 250 kDa, 300 kDa, 350 kDa, 400 kDa, 450 kDa, 500 kDa, 600 kDa, 700 kDa, 800 kDa, 900 kDa, 1000 kDa, or more, or any range bracketed by any of the values listed. In certain embodiments, the payload molecules other than the smallest payload molecules have a molecular weight of at least about 2 kDa, 3 kDa, preferably at least about 5 kDa, 10 kDa, 15 kDa, 20 kDa, 25 kDa, 30 kDa, 35 kDa, 40 kDa, 50 kDa, 60 kDa, 70 kDa, 80 kDa, 90 kDa, 100 kDa, 110 kDa, 120 kDa, 130 kDa, 140 kDa, 150 kDa, 160 kDa, 170 kDa, 180 kDa, 190 kDa, 200 kDa, 250 kDa, 300 kDa, 350 kDa, 400 kDa, 450 kDa, 500 kDa, 600 kDa, 700 kDa, 800 kDa, 900 kDa, 1000 kDa, or more, or any range bracketed by any of the values listed.

In certain embodiments, at least one payload comprises polyethylene glycol (PEG). It is understood that PEG polymers are typically commercially provided as mixtures of PEG polymers with an average molecular weight or within a range of molecular weights. As used herein, a PEG payload for example, of about at least 5 kDa is understood as a payload attached to a tether wherein the PEG prior to attachment to the tether as provided by a commercial source is indicated to substantially have a molecular weight at least 5 kDa (e.g., at least 80%, at least 85%, at least 80%, at least 90%, at least 95%, at least 98%, at least 99% of the PEG molecules having a molecular weight of at least 5 kDa). In reference to polymers, molecular weights can be understood as the average molecular weight of the polymers as provided from commercial sources.

In certain embodiments, the payload comprises a therapeutic agent. In certain embodiments, the therapeutic agent is selected from the group consisting of doxorubicin (DOXO), 6-mercaptopurine, 6-thioguanine, cytarabine, 5-fluorouracil decarbazine, mechlorethamine, thioepa chlorambucil, CC-1065, melphalan, carmustine (BSNU), lomustine (CCNU), cyclothosphamide, busulfan, dibromomannitol, streptozotocin, mitomycin, cis-dichlorodiamine platinum (II) (DDP) cisplatin, daunorubicin, dactinomycin, bleomycin, mithramycin, anthramycin (AMC), vincristine, vinblastine, taxol, paclitaxel, maytansinoids, cytochalasin B, gramicidin D, ethidium bromide, emetine, etoposide, tenoposide, colchicin, dihydroxy anthracin dione, mitoxantrone, mithramycin, 1-dehydrotestosterone, glucocorticoids, procaine, tetracaine, lidocaine, propranolol, puromycin, and calicheamicin.

In certain embodiments, the payload comprises a detectable label. In certain embodiments, the detectable label is selected from the group consisting of enzymatic label, fluorescent label, radioactive label, dense particle label, chemiluminescent label, bioluminescent label, prosthetic group label, fluorescence emitting metal atom, radioactive isotope, microparticle, nanoparticle, quantum dot, electron-dense reagent, hapten, biotin, streptavidin, avidin, and neutravidin.

In certain embodiments, the payload comprises a nucleic acid. In certain embodiments, the nucleic acid is selected from the group consisting of DNA, RNA, LNA, PNA, microRNA, chimeric nucleic acid, modified nucleic acid, single stranded nucleic acid, double stranded nucleic acid, aptamer, and chemically modified nucleic acid.

In certain embodiments, the tether comprises a nanopolymer. In certain embodiments, the tether is selected from the group consisting of polylysine, polyglutamic acid, N-(2-hydroxypropyl)methacrylamide, polycation polymers, poly(allylamine), poly(dimethyldiallyammonim chloride) polylysine, poly(ethylenimine), poly(allylamine), natural polycations, dextran amine, polyarginine, chitosan, gelatine A, protamine sulfate, polyanion polymers, poly(styrenesulfonate), polyglutamic or alginic acids, poly(acrylic acid), poly(aspartic acid), poly(glutaric acid), natural polyelectrolytes with similar ionized groups, dextran sulfate, carboxymethyl cellulose, hyaluronic acid, sodium alginate, gelatine B, chondroitin sulfate, and heparin.

In certain embodiments, the mixed payload polymer does not comprise diethylene triaminepentaacetic acid (DTPA). In certain embodiments, the bispecific ligand does not bind DTPA.

In certain embodiments, the capturing tag is selected from biotin, DTPA, 6× histidine, hemagulutinin tag, and myc tag.

In certain embodiments, the mixed payload polymer further comprises a biotin moiety that is not a payload molecule. In certain embodiments, the non-payload biotin containing mixed payload polymer is linked to at least one additional mixed payload polymer. In certain embodiments, the mixed payload polymer further comprises an avidin moiety selected from the group consisting of avidin, strepavidin, and neutravidin, wherein the avidin moiety is not a payload molecule.

In certain embodiments, the mixed payload polymer is not succinylated. In certain embodiments, the mixed payload polymer is succinylated. In certain embodiments, the mixed payload polymer is modified with a non-payload molecule to alter charge or solubility of the polymer.

In certain embodiments, the payload is not biotin. In certain embodiments, the payload is biotin. In certain embodiments, the payload is not an avidin moiety. In certain embodiments, the payload is an avidin moiety. In certain embodiments, the payload is not a wild-type mammalian protein.

In certain embodiments, the payload comprises one or more therapeutic agents. In certain embodiments, the payload comprises one or more in vivo diagnostic agents. In certain embodiments, the payload comprises one or more therapeutic agents. In certain embodiments, the payload comprises one or more therapeutic agents and one or more in vivo diagnostic agents. In certain embodiments, the payload comprises one or more in vitro detectable labels.

In certain embodiments, the payload molecules are all the same. In certain embodiments, the payload molecules comprise two or more distinct payload molecules.

In a preferred embodiment, the two or more distinct payload molecules have about the same molecular weight. For example, the molecular weight of the largest payload is no more than 5-fold greater than the molecular weight of the smallest payload molecule. Alternatively, or additionally, the molecular weight of the payload molecules varies no more than 50% from the average molecular weight of all of the payload molecules.

In certain embodiments, the first binding site of the bispecific ligand is comprised in a molecule selected from the group consisting of antibody, antibody fragment, antibody mimetic, e.g., affibody, T-cell receptor, nucleic acid, hapten, hormone, cytokine, receptor, receptor ligand, therapeutic agent, chelator, heavy metal, polypeptide, small molecule, hormone, cytokine, therapeutic agent, imaging agent, detectable label, antigen, receptor, biotin, streptavidin, avidin, and neutravidin.

In certain embodiments, the second binding site of the bispecific ligand is comprised in a molecule selected from the group consisting of antibody, antibody fragment, antibody mimetic, e.g., affibody, T-cell receptor, nucleic acid, hapten, hormone, cytokine, receptor, receptor ligand, therapeutic agent, chelator, heavy metal, polypeptide, small molecule, hormone, cytokine, therapeutic agent, imaging agent, detectable label, antigen, receptor, biotin, streptavidin, avidin, and neutravidin.

In certain embodiments, the first binding site of the bispecific ligand and the second binding site of the bispecific ligand are selected independently.

In certain embodiments, one or both of the first binding site of the bispecific ligand and the second binding site of the bispecific ligand are comprised of an antibody or antibody fragment, each of which is independently selected from the group consisting of IgG, IgM, IgA1, IgA2, IgD, IgE, polyclonal antibody, monoclonal antibody, modified antibody, chimeric antibody, reshaped antibody, antibody mimetic, e.g., affibody, humanized antibody, Fab fragment, a F(ab′)2 fragment, a Fd fragment, a Fv fragment, a dAb fragment, single chain Fv, and one or more isolated complementarity determining regions (CDR) that retain specific binding to its cognate antigen.

In certain embodiments, one or both of the first binding site of the bispecific ligand and the second binding site of the bispecific ligand are comprised of a nucleic acid, each of which is independently selected from the group consisting of DNA, RNA, PNA, modified DNA, modified RNA, microRNA, LNA, mixed nucleic acid polymer, aptamer, and ribozyme.

In certain embodiments, one or both of the first binding site of the bispecific ligand and the second binding site of the bispecific ligand are comprised of a hormone selected from the group consisting of insulin, estrogen, progestin, progesterone, human growth hormone, melatonin, serotonin, thyroxine, triiodothyronine, epinephrine, norepinephrine, dopamine, antimullerian hormone, adiponectin, adreoncorticotropic hormone, angiotensin, vasopressin, atripeptin, calcitonin, cholecystokinin, corticotrophin releasing hormone, erythropoietin, follicle-stimulating hormone, gastrin, ghrelin, glucagon, gonadotropin releasing hormone, growth hormone releasing hormone, human chorionic gonadotropin, human placental lactogen, growth hormone, inhibin, insulin, insulin-like growth factor, leptin, lutenizing hormone, melanocyte stimulating hormone, orexin, ozytocin, parathyroid hormone, prolactin, relaxin, secretin, somatostatiin, thrombopoietin, thyroid stimulating hormone, thyrotropin-releasing hormone, cortisol, aldosterone, testosterone, dehydroepiandrosterone, androstenedoine, dihydrotestosterone, estradiol, estrone, estriol, progesterone, calcitrol, calcidiol, prostaglandings, leukotrienes, prostacyclin, thomboxane, prolactin releasing hormone, lipotropin, brain natriuretic peptide, neuropeptide Y, histamine, endothelin, pancreatic polypeptide, rennin, and enkephalin.

In certain embodiments, one or both of the first binding site of the bispecific ligand and the second binding site of the bispecific ligand are comprised of a cytokine selected from the group consisting of a lymphokine, an interleukin, and a chemokine.

In certain embodiments, one or both of the first binding site of the bispecific ligand and the second binding site of the bispecific ligand are comprised of a therapeutic agent selected from the group consisting of doxorubicin (DOXO), 6-mercaptopurine, 6-thioguanine, cytarabine, 5-fluorouracil decarbazine, mechlorethamine, thioepa chlorambucil, CC-1065, melphalan, carmustine (BSNU), lomustine (CCNU), cyclothosphamide, busulfan, dibromomannitol, streptozotocin, mitomycin, cis-dichlorodiamine platinum (II) (DDP) cisplatin, daunorubicin, paclitaxel, dactinomycin, bleomycin, mithramycin, anthramycin (AMC), vincristine, vinblastine, taxol, maytansinoids, cytochalasin B, gramicidin D, ethidium bromide, emetine, etoposide, tenoposide, colchicin, dihydroxy anthracin dione, mitoxantrone, mithramycin, 1-dehydrotestosterone, glucocorticoids, procaine, tetracaine, lidocaine, propranolol, puromycin, and calicheamicin.

In certain embodiments, the bispecific ligand does not bind biotin. In certain embodiments, the bispecific ligand does bind to biotin. In certain embodiments, the bispecific ligand does not bind to an avidin moiety. In certain embodiments, the bispecific ligand does bind to an avidin moiety. In certain embodiments, the bispecific ligand does not bind DPTA.

In certain embodiments, the first binding site binds to PEG. In certain embodiments, the second binding site does not bind to PEG. In certain embodiments, the first binding site binds to a PEG molecule that has been modified, for example, for attachment to a tether. As provided herein, a PEG payload must have a PEG epitope that can be bound by a PEG-specific antibody.

In certain embodiments, the first binding site of the bispecific ligand binds a payload wherein each payload molecule has a molecular weight, or an average molecular weight, of at least 2 kDa, 3 kDa, preferably at least 5 kDa, more preferably at least 10 kDa, 15 kDa, 20 kDa, 25 kDa, 30 kDa, 35 kDa, 40 kDa, 50 kDa, 60 kDa, 70 kDa, 80 kDa, 90 kDa, 100 kDa, 110 kDa, 120 kDa, 130 kDa, 140 kDa, 150 kDa, 160 kDa, 170 kDa, 180 kDa, 190 kDa, 200 kDa, 250 kDa, 300 kDa, 350 kDa, 400 kDa, 450 kDa, 500 kDa, 600 kDa, 700 kDa, 800 kDa, 900 kDa, 1000 kDa, or more, or any range bracketed by any of the values listed, and the second binding site of the bispecific ligand binds a target wherein the target has a molecular weight of at least 5 kDa, at least 10 kDa, at least 15 kDa, at least 20 kDa, at least 25 kDa, at least 30 kDa, at least 40 kDa, at least 50 kDa, at least 60 kDa, or at least 75 kDa.

In certain embodiments, the second binding site of the bispecific ligand binds specifically to a target that is present in a sample. In certain embodiments, the target present in the sample is endogenous to the sample. In certain embodiments, the target present in the sample binds specifically to a compound that is endogenous to the sample.

In certain embodiments, the bispecific ligand is a regiospecific bispecific ligand.

In certain embodiments, the bispecific ligand includes a third binding site.

In certain embodiments, the contacting comprises contacting in vitro. Contacting is performed under conditions to permit binding which can be determined by one of skill in the art based on the binding partners.

In certain embodiments, the target molecule is attached to a solid support. In certain embodiments, the solid support is selected from the group consisting of ELISA plate, tissue/cell sample, microscope slide, beads, nanoparticles, and microarrays.

In certain embodiments, the method comprises detecting the target or molecule for detection. In certain embodiments, detecting the target comprises quantitatively detecting the target. In certain embodiments, the limit of detection in vitro is about 500 ng/ml, 400 ng/ml, 300 ng/ml, 250 ng/ml, 200 ng/ml, 150 ng/ml, 100 ng/ml, 75 ng/ml, 50 ng/ml, 40 ng/ml, 30 ng/ml, 25 ng/ml, 20 ng/ml, 15 ng/ml, 10 ng/ml, 5 ng/ml, 1 ng/ml, 0.1 ng/ml, 0.01 ng/ml, or 0.001 ng/ml or less, or 10⁻¹³ g/ml or less, or 10⁻¹⁴ g/ml or less, or 10⁻¹⁵ g/ml or less, or 10⁻¹⁶ g/ml or less, or 10⁻¹⁷ g/ml or less, or 10⁻¹⁸ g/ml or less, or any range bracketed by any two of the values provided. In certain embodiments, the payload comprises a detectable label. In certain embodiments, the target or molecule for detection is present at a concentration of about 10 pM, 1 pM, 100 fM, 10 fM, 1 fM, 100 aM, 10 aM, 1 aM, 100 zM, 10 zM, 1 zM or less; or any range bracketed by any of the values provided.

The invention provides methods of detecting a target nucleic acid sequence in a sample, wherein the target nucleic acid sequence is detected without nucleic acid amplification.

In certain embodiments, the number of copies of the target nucleic acid sequence in the sample is about 50,000 copies, 10,000 copies, 7500 copies, 5000 copies, 2500 copies, 1000 copies, 500 copies, or less; or any range bracketed by any of the values provided. In certain embodiments, the target nucleic acid sequence is present at a concentration of about 10 pM, 1 pM, 100 fM, 10 fM, 1 fM, 100 aM, 10 aM, 1 aM, 100 zM, 10 zM, 1 zM or less; or any range bracketed by any of the values provided.

In certain embodiments, the detectable label is detected directly. In certain embodiments, the detectable label is detected by contacting the detectable label with at least one other reagent. In certain embodiments, the at least one other reagent comprises a molecule selected from the group consisting of enzymatic label, fluorescent label, radioactive label, dense particle label, chemiluminescent label, bioluminescent label, prosthetic group label, fluorescence emitting metal atom, radioactive isotope, quantum dot, microparticle, nanoparticle, electron-dense reagent, and hapten; wherein the molecule further comprises a moiety selected from the group consisting of biotin, streptavidin, avidin, and neutravidin. In certain embodiments, the at least one other reagent comprises an enzymatic label or a fluorescent label and a biotin moiety. In certain embodiments, the detectable label is contacted with an enzyme substrate.

In certain embodiments, the contacting comprises contacting in a subject in vivo.

In certain embodiments, the method comprises delivering a therapeutic agent to the target or molecule for detection. In certain embodiments, the method comprises delivering an imaging agent to the target or molecule for detection. In certain embodiments, the method comprises a therapeutic method.

In certain embodiments, the method comprises detecting the target or molecule for detection. In certain embodiments, detecting the target or molecule for detection comprises quantitatively detecting the target or molecule for detection. In certain embodiments, detecting the target comprises detecting the location of the target in the subject.

In certain embodiments, the method comprises a diagnostic method. In certain embodiments, the method comprises an in vivo diagnostic method. In certain embodiments, the method comprises an in vitro diagnostic method.

In certain embodiments, the target or molecule for detection is endogenous to the sample or subject. In certain embodiments, the target or molecule for detection is not endogenous to the sample or subject. In certain embodiments wherein the target or molecule for detection is not endogenous to the subject sample includes a compound that specifically binds to a target endogenous to the subject sample.

In certain embodiments, the method comprises the use of a mixed payload polymer.

In certain embodiments, the method comprises the use of a bispecific ligand.

The invention provides mixed payload polymers comprising a tether molecule linked to two or more payload molecules, wherein the payload molecules attached to the tether have at least 50% of the activity of a molar equivalent of payload molecules not attached to a tether.

The invention provides compositions for use in the methods of the invention.

The invention provides populations of mixed payload polymers, wherein each of the payload molecules are present at a molar ratio to the tethers at a ratio of at least 1.5:1. In certain embodiments, the molar ratio of each of the payload molecules to the tether is independently selected from about 500:1, 450:1; 400:1; 350:1, 300:1, 250:1, 200:1, 150:1, 100:1, 50:1; 25:1, 20:1, 19:1, 18:1, 17:1, 16:1, 15:1, 14:1, 13:1, 12:1, 11:1, 10:1, 9:1, 8:1, 7:1, 6:1, 5:1, 4:1, 3:1, 2.5:1; 2:1, or any range bracketed by the values provided. In certain embodiments, the molar ratio of the total number of payload molecules to the tether is about 500:1, 450:1; 400:1; 350:1, 300:1, 250:1, 200:1, 150:1, 100:1, 50:1; 25:1, 20:1, 19:1, 18:1, 17:1, 16:1, 15:1, 14:1, 13:1, 12:1, 11:1, 10:1, 9:1, 8:1, 7:1, 6:1, 5:1, 4:1, 3:1, 2.5:1; 2:1, or any range bracketed by the values provided. It is understood that although a single tether will necessarily have a whole number of payload molecules. However, within a population of tethers, on average, the population can have something other than a whole number of each type of payload molecule, e.g., 1.5, 2.5, 3.5, etc. In certain embodiments, in a population of tethers, at least 40%, 50%, 60%, 70%, 80%, 85%, 90%, 95%, or 98% of the tethers or any range bracketed by the values provided, include at least 2 payload molecules.

The invention provides bispecific ligands comprising a first binding site and a second binding site wherein the first binding site binds specifically to a payload molecule and the second binding site binds specifically to a target that is not the payload molecule bound by the first site. In certain embodiments, the payload molecule bound by the bispecific ligand is PEG.

The invention provides and includes any reagents specific for practicing the methods of the invention.

The invention provides kits including mixed payload polymers provided herein wherein the kits comprise a payload, and a bispecific ligand provided herein comprising a first binding site and a second binding site wherein the first binding site binds specifically to the payload and the second binding site binds specifically to a target that is not the payload. In certain embodiments, the kit comprises a reagent for detection of the payload.

The invention further provides kits including a mixed payload polymer that includes a payload, a first molecule with a binding site that specifically binds to the payload on the mixed payload polymer, and one, two, or all three of
a) a reagent for covalently linking the first molecule comprising a binding site to a second molecule comprising a binding site;

b) a device for separating a covalently linked first molecule comprising a binding site and a second molecule comprising a binding site from a non-covalently linked first molecule comprising a binding site and a non-covalently linked second molecule comprising a binding site; and
c) a reagent for detecting a payload molecule.

In certain embodiments, the reagent for covalently linking the first molecule with a binding site to a second molecule with a binding site is covalently linked to the first molecule in the kit. In certain embodiments, the device for separating a covalently linked first molecule comprising a binding site and a second molecule comprising a binding site from a non-covalently linked first molecule comprising a binding site and a non-covalently linked second molecule comprising a binding site is selected from the group consisting of a dialysis membrane, a spin column, a gravity flow column, size exclusion chromatography column, and a gel filtration chromatography column.

In certain embodiments, the kits of the invention include a reagent for detection of the payload.

In certain embodiments, the reagent for detection of the payload comprises a molecule selected from the group consisting of enzymatic label, fluorescent label, radioactive label, dense particle label, chemiluminescent label, bioluminescent label, prosthetic group label, fluorescence emitting metal atom, radioactive isotope, quantum dot, nanoparticle, electron-dense reagent, and hapten; wherein the molecule further comprises a moiety selected from the group consisting of biotin, streptavidin, avidin, and neutravidin. In certain embodiments, the reagent comprises an enzymatic label or a fluorescent label and a biotin moiety. In certain embodiments, the detectable label is contacted with an enzyme substrate.

Other embodiments are provided infra.

### BRIEF DESCRIPTION OF THE DRAWINGS

FIGS. 1A-C schematically show the formation of (A) primary, (B) secondary, and (C) tertiary complexes formed using in vitro detection methods provided herein in combination with the payload containing tethers and bispecific ligands provided herein.

FIG. 2 schematically shows the formation of a complex using a bispecific ligand including a receptor binding ligand and an antibody for the targeting of a payload to a cell.

FIG. 3 schematically shows the formation of a complex using a bispecific ligand including a nucleic acid ligand and an antibody for targeting the payload to the nucleic acid sequence.

FIG. 4 schematically shows a multimerized mixed payload polymers.

FIG. 5 schematically shows a complex including a bispecific ligand including a biotin-binding site, mixed payload polymer with biotin as a payload, and a reagent for detecting the biotin bound to the target through the complex.

FIG. 6 is a graph of detection of myosin heavy chain fragments with payload containing polymers with an average of 1.5, 3, 4.5, 6, and 7.5 horseradish peroxidase molecules per tether.

FIG. 7 shows graphs comparing the results from assays performed using three different HRP-linked detection reagents.

FIG. 8 shows graphs comparing the results from assays performed using anti-DTPA, anti-PEG, and anti-HRP antibodies to capture the same mixed payload polymer containing DTPA, PEG, and HRP payloads.

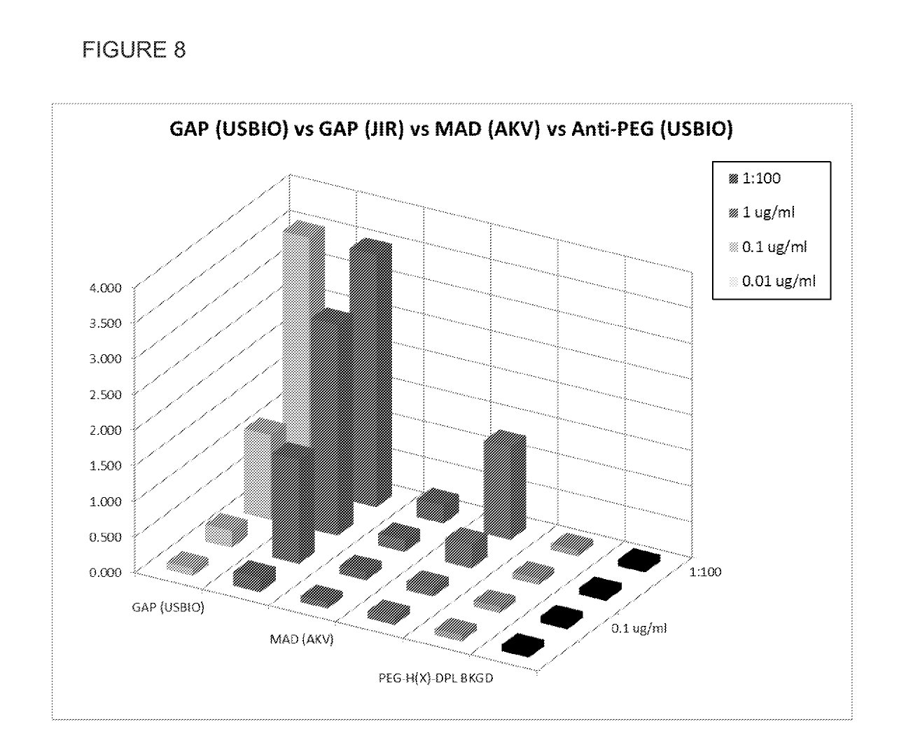

### DETAILED DESCRIPTION OF THE INVENTION

### Definitions

As used herein, each of the following terms has the meaning associated with it in this section.

The terms “administer”, “administering” or “administration” include any method of delivery of a pharmaceutical composition or agent into a subject's system or to a particular region in or on a subject. In certain embodiments of the invention, the agent is administered topically. In certain embodiments of the invention, an agent is administered intravenously, intramuscularly, subcutaneously, intrathecally, intracereberal, intraventricular, intraspinal, intradermally, intranasally, orally, transcutaneously, or mucosally.

As used herein, “antibody” is understood as a protein that includes at least one complementary determining region that binds to a specific target antigen. An antibody frequently includes at least one immunoglobulin variable region, e.g., an amino acid sequence that provides an immunoglobulin variable domain or immunoglobulin variable domain sequence. For example, an antibody can include a heavy (H) chain variable region (abbreviated herein as VH), and a light (L) chain variable region (abbreviated herein as VL). In another example, an antibody includes two heavy (H) chain variable regions and two light (L) chain variable regions. The term “antibody” encompasses antigen-binding fragments of antibodies (e.g., single chain antibodies, Fab, F(ab′)2, Fd, Fv, and dAb fragments) as well as complete antibodies, e.g., intact immunoglobulins of types IgA, IgG, IgE, IgD, IgM (as well as subtypes thereof). The light chains of the immunoglobulin can be of types kappa or lambda. In one embodiment, the antibody is glycosylated. For example, an antibody can be a polyclonal antibody, a monoclonal antibody, a modified antibody, a chimeric antibody, a reshaped antibody, a humanized antibody, a Fab fragment, a F(ab′)2 fragment, a Fd fragment, a Fv fragment, a dAb fragment, single chain Fv, a dimerized variable region (V region) fragment (diabody), a disulfide-stabilized V region fragment (dsFv), an affibody, an antibody mimetic, and one or more isolated complementarity determining regions (CDR) that retain specific binding to their cognate antigen. As used herein, an “isolated” CDR is a CDR not in the context of a naturally occurring antibody. The antibody can be any immunoglobulin type, e.g., IgG, IgM, IgA1, IgA2, IgD, or IgE.

As used herein, an “antigen” is any molecule that can be specifically bound by an antibody. An “antigen” can be an “endogenous antigen”, i.e., naturally occurring, or potentially naturally occurring, antigen in the subject or sample to which a payload is to be delivered, e.g., a wild-type protein, a mutant protein, a tumor specific antigen; or a “non-endogenous antigen” i.e., not a naturally occurring antigen in the subject or sample to which a payload is to be delivered, e.g., a detectable label, e g, alkaline phosphatase, horse radish peroxidase, biotin, present in a payload molecule to be delivered to the subject or sample.

The term “antigen-binding portion” of an antibody (or simply “antibody portion”), as used herein, refers to one or more fragments of an antibody that retain the ability to bind specifically to an antigen. It has been shown that the antigen-binding function of an antibody can be performed by fragments of a full-length antibody. Such antibody embodiments may also be bispecific, dual specific, or multi-specific formats—specifically binding to two or more different antigens. Examples of binding fragments encompassed within the term “antigen-binding portion” of an antibody include (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; demonstrating the sufficiency of a disulfide bond to mediate dimerization (iii) a Fd fragment consisting of the VH and CH1 domains; (iv) a Fv fragment consisting of the VL and VH domains of a single arm of an antibody, (v) a dAb fragment (Ward (1989) Nature 341: 544-546; and PCT Publication No. WO 90/05144 A1), which comprises a single variable domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242: 423-426; and Huston et al. (1988) Proc. Natl. Acad. Sci. USA 85: 5879-5883). Such single chain antibodies are also intended to be encompassed within the term “antigen-binding portion” of an antibody. Other forms of single chain antibodies, such as diabodies, are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see, e.g., Holliger, P. et al. (1993) Proc. Natl. Acad. Sci. USA 90: 6444-6448; Poljak, R. J. et al. (1994) Structure 2: 1121-1123). Such antibody binding portions are known in the art (Kontermann and Dubel eds., Antibody Engineering (2001) Springer-Verlag. New York. pp. 790 (ISBN 3-540-41354-5)). In addition single chain antibodies also include “linear antibodies” comprising a pair of tandem Fv segments (VH-CH1-VH-CH1) that, together with complementary light chain polypeptides, form a pair of antigen binding regions (Zapata et al. (1995) Protein Eng. 8(10): 1057-1062 and U.S. Pat. No. 5,641,870).

As used herein, a “binding site” can be at least a portion of a molecule that can specifically bind a target molecule in vivo or in vitro. Binding sites can be at least a portion of or comprised in other molecules such as an antibody, antibody fragment, antibody mimetic, nucleic acid, hapten (e.g., biotin, PEG), streptavidin, avidin, neutravidin, hormone, cytokine, receptor ligand, carbohydrate, and therapeutic agent. In certain embodiments, the binding site specifically binds to a molecule that is present in the sample or subject to which the payload is to be delivered. In certain embodiments, the binding site does not specifically bind to a molecule that is present in the sample or subject to which the payload is to be delivered. In certain embodiments, the binding site specifically binds to the payload. In certain embodiments, the binding site does not specifically bind to the payload.

As used herein, a “bispecific ligand” is understood as a molecule that comprises two specific binding sites for binding two distinct molecules wherein the bispecific ligand can specifically bind both molecules simultaneously. It is understood that a bispecific ligand can include more than two binding sites as long as the ligand includes at least one binding site for each of two ligands. In certain embodiments, bispecific ligands include only two binding sites. Bispecific ligands act as targeting agents, bringing the payload to the site of interest. Bispecific ligands can include, but are not limited to formats such as “Bispecific Antibody-Antibody” or “BAB”; “Bispecific Antibody-Ligand” or “BAL”; “Bispecific Antibody-Molecular Probe” or “BAMP”; or a “Bispecific-Avidin/Biotin Ligand”. In certain embodiments, the binding sites are joined to each other in specific relative orientations, i.e., joined with a regiospecific linkage. In certain embodiments, a bispecific ligand can also be known as a capturing agent. In certain embodiments, the bispecific ligands include two or more binding sites. In certain embodiments, the bispecific ligands include only two binding sites. In certain embodiments, bispecific ligands are in populations in which at least 80%, at least 85%, at least 90%, at least 95%, at least 97%, at least 98%, or at least 99% of the bispecific ligands have only two binding sites.

As used herein, a “Bispecific Antibody-Antibody” or “BAB” is understood as a bispecific ligand comprised of two antibodies, or fragments or mimetics thereof, joined to each other wherein one antibody specifically binds a target, and the other antibody specifically binds to the tether, e.g., by binding directly to the tether; or indirectly to the tether by binding to a tag or to the payload on the tether.

As used herein, a “Bispecific Antibody-Ligand” or “BAL” is understood as a bispecific ligand having a binding site not comprised in an antibody or a nucleic acid (e.g., a drug, a non-peptide hormone, etc.), joined to an antibody. In certain embodiments in the BAL, the ligand binds specifically to a target, and the antibody binds either directly or indirectly to the tether. In other embodiments in the BAL, the antibody binds specifically to a target, and the ligand binds either directly or indirectly to the tether.

As used herein, a “Bispecific Antibody-Molecular Probe” or “BAMP” as used herein is understood as a bispecific ligand comprising a binding site that includes any molecule, natural or synthetic, which is made of nucleic acids or nucleic acid mimetics, which include, but are not limited to, a DNA, an RNA, a mixed nucleic acid (e.g., DNA-RNA hybrid), microRNA, LNA, PNA, a modified nucleic acid, e.g., phosphorothioate containing, 2′-modified sugar containing, a primer, an oligomer, or any other molecule that specifically binds to a target site, and joined to an antibody, wherein the nucleic acid binds specifically to a target, by base pairing or structure recognition, and the antibody binds either directly or indirectly to the tether.

As used herein, a “Bispecific-Avidin/Biotin Ligand” is a bispecific ligand in which one of the binding sites is an avidin (e.g., avidin, streptavidin, neutravidin) or biotin; and the other binding site binds to a target molecule in a sample. A bispecific-avidin/biotin ligand is a type of BAL. In preferred embodiments, one binding sites comprises an avidin, preferably streptavidin. In certain embodiments, the other binding site comprises an antibody, a ligand, or a nucleic acid. In certain embodiments, the other binding site binds to a target molecule endogenous to the sample. In certain embodiments, the other binding site binds to an agent that is not endogenous to the sample. A “Bispecific-Avidin/Biotin Ligand” is used in capture an avidin (e.g., avidin, streptavidin, neutravidin) or biotin payload-containing tether, preferably a biotin payload-containing tether.

“Biotin/Streptavidin System” is understood as a detection system based on the combined use of biotin with streptavidin. “Biotin” is a small molecule which can be conjugated, for example, to a detection or secondary antibody or a tether. Biotin has very high binding affinity for avidin moieties, e.g., streptavidin avidin, or neutravidin, and is useful as a detection reagent across a number of platforms. “Streptavidin” or “SA” is a protein, can be conjugated to one or several detectable labels such as horseradish peroxidase (HRP), to join the biotin containing molecule to the streptavidin containing molecule with very high binding affinity. It is understood that streptavidin may be substituted with avidin or neutravidin.

As used herein, “capture reagent”, “capture antibody” or “CAB” is understood as a reagent, e.g., an antibody, extracellular matrix components, a cell, that is coated onto a solid support that is used to capture a target antigen. Antibody, sometimes coated on a plastic plate or well, polymer beads, nanoparticles, or other support matrix, which is used to capture a targeted antigen. In certain embodiments, the capture reagent can be present on the cell, e.g., a cell surface receptor.

As used herein, a “chelator” is understood as a compound that binds a ligand, such as a heavy metal (e.g., mercury, arsenic, or lead) or cation (e.g., Cu²⁺, Mg²⁺, Ca²⁺). These groups include a number of radiolabels used in detection or radiotherapy methods. Chelators bind to their ligands by the formation of two or more separate coordinate bonds between the chelator and the ligand. Chelation of the metal or cation inactivates the metal or cation so it cannot react with other elements or ions to have its usual effect. Chelators include, but are not limited to, EDTA, EGTA, methylamine, histidine, malate, phytochelatin, polyphosphanates, bidentate phosphine, or metal binding proteins.

The term “control sample,” as used herein, refers to any clinically or scientifically relevant comparative sample, including, for example, a sample from a healthy subject, a sample from a subject having a deficiency that can cause or make the subject susceptible to a certain disease or condition, a subject with a disease or condition of interest, a sample from a subject treated with a pharmaceutical carrier, a sample from a subject prior to treatment, a sham or buffer treated subject or sample, an untreated subject or sample, and the like.

The term “control level” refers to an accepted or pre-determined level of a biological marker, e.g., a level of a marker obtained before treatment or the onset of disease. The level of a biological marker present in a subject or population of subjects having one or more particular characteristics, e.g., the presence or absence of a particular disease or condition.

As used herein, “changed as compared to a control” sample or subject is understood as having a level of the analyte or diagnostic or therapeutic indicator (e.g., marker) to be detected at a level that is statistically different than a sample from a normal, untreated, or control sample. Control samples include, for example, cells in culture, one or more laboratory test animals, or one or more human subjects. Methods to select and test control samples are within the ability of those in the art. An analyte can be a naturally occurring substance that is characteristically expressed or produced by the cell or organism (e.g., an antibody, a protein), a substance produced by a reporter construct (e g, alkaline phosphatase, β-galactosidase or luciferase). Depending on the method used for detection the amount and measurement of the change can vary. Changed as compared to a control reference sample can also include a change in one or more signs or symptoms associated with or diagnostic of disease. Determination of statistical significance is within the ability of those skilled in the art, e.g., the number of standard deviations from the mean that constitute a positive result.

The term “cytokine” is a generic term for proteins released by one cell population, which act on another cell population as intercellular mediators. Examples of such cytokines are lymphokines, monokines, and traditional polypeptide hormones. Included among the cytokines are growth hormone, such as human growth hormone, N-methionyl human growth hormone, and bovine growth hormone; parathyroid hormone; thyroxine; insulin; proinsulin; relaxin; prorelaxin; glycoprotein hormones, such as follicle stimulating hormone (FSH), thyroid stimulating hormone (TSH), and luteinizing hormone (LH); hepatic growth factor; fibroblast growth factor; prolactin; placental lactogen; tumor necrosis factor-alpha and -beta; mullerian-inhibiting substance; mouse gonadotropin-associated peptide; inhibin; activin; vascular endothelial growth factor; integrin; thrombopoietin (TPO); nerve growth factors, such as NGF-alpha; platelet-growth factor; placental growth factor, transforming growth factors (TGFs), such as TGF-alpha and TGF-beta; insulin-like growth factor-1 and -11; erythropoietin (EPO); osteoinductive factors; interferons, such as interferon-alpha, -beta and -gamma; colony stimulating factors (CSFs), such as macrophage-CSF (M-CSF), granulocyte macrophage-CSF (GM-CSF), and granulocyte-CSF (G-CSF); interleukins (ILs), such as IL-1, IL-2, IL-3, IL-4, IL-5, IL-6, IL-7, IL-8, IL-9, IL-10, IL-11, IL-12, IL-13, IL-15, IL-18, IL-21, IL-22, IL-23, and IL-33; a tumor necrosis factor, such as TNF-alpha or TNF-beta; and other polypeptide factors including LIF and kit ligand (KL). As used herein, the term cytokine includes proteins from natural sources or from recombinant cell culture and biologically active equivalents of the native sequence cytokines.

As used herein, “detecting”, “detection” and the like are understood that an assay performed for identification of a specific analyte or activity in a sample. The amount of analyte or activity detected in the sample can be none or below the level of detection of the assay or method.

As used herein, a “detectable label” is understood as a molecule that can be detected, preferably quantitatively, when present in a sample or subject, including, but not limited to, enzymatic label (e.g., alkaline phosphatase), fluorescent label, radioactive label, dense particle label, chemiluminescent label, bioluminescent label, prosthetic group label, fluorescence emitting metal atom, radioactive isotope, quantum dot, nanoparticle, electron-dense reagent, hapten, PEG, or biotin. In certain embodiments, detectable labels, e.g., fluorescent label, radioactive label, dense particle label, chemiluminescent label, bioluminescent label, fluorescence emitting metal atom, radioactive isotope, quantum dot, electron-dense reagent, can be encapsulated in microparticles, nanoparticles, lipidots, micells, liposomes, or other similar carriers. Those of skill in the art will understand that the specific detectable label for use in a particular method will be determined, for example, on the target to be detected, the sample in which the detection is performed (e.g., liquid or solid sample, detection in vitro or in vivo), and the equipment available for detection. A detectable label can be detected directly, e.g., a fluorescent label. Alternatively, a detectable label can be detected by contacting the detectable label with at least one additional reagent, e.g., an enzyme substrate that produces a color, fluorescent, or luminescent product; a reagent that binds the mixed payload polymer, e.g., an avidin containing label for binding a biotin-containing tether, a nickel containing tether for binding 6×His; that is detected directly, e.g., a fluorescent or radioactive label, or indirectly, e.g., an enzyme.

“Determining” as used herein is understood as performing an assay or using a diagnostic method to ascertain the state of someone or something, e.g., the presence, absence, level, or degree of a certain condition, biomarker, disease state, or physiological condition.

By “diagnosing” and the like, as used herein, refers to a clinical or other assessment of the condition of a subject based on observation, testing, or circumstances for identifying a subject having a disease, disorder, or condition based on the presence of at least one indicator, such as a sign or symptom of the disease, disorder, or condition. Diagnostic methods provide an indicator that a disease is or is not present. A single diagnostic test typically does not provide a definitive conclusion regarding the disease state of the subject being tested. Diagnostic agents for use in vivo include any clinically acceptable agent that can be detected in a human using imaging methods such as MRI, CAT scan, CT scan, bone scan, x-ray, and ultrasound. Diagnostic agents for use in humans include, but are not limited to heavy metal, dense particle, nanoparticle, microparticle, spin label, and radiolabels such as technetium (⁹⁹mTc), and gallium (⁶⁷Ga).

As used herein, an “enzymatic label” is understood as a molecule, typically a protein, that converts a substrate into a detectable product or label, e.g., a fluorescent label, chromophore label, preferably in a quantitative manner through at least a concentration range of two or more orders of magnitude. Enzymatic labels include, but are not limited to, alkaline phosphatase, beta-galactosidase, luciferase, and horse radish peroxidase.

The term “expression” is used herein to mean the process by which a polypeptide is produced from DNA. The process involves the transcription of the gene into mRNA and the translation of this mRNA into a polypeptide. Depending on the context in which used, “expression” may refer to the production of RNA (including micro RNAs and siRNAs) or protein or both.

The terms “level of expression of a gene” or “gene expression level” refer to the level of mRNA, as well as pre-mRNA nascent transcript(s), transcript processing intermediates, mature mRNA(s) and degradation products, or the level or activity of protein encoded by the gene in the cell or tissue.

As used herein, a “fluorescent label” is understood as a detectable label including a fluorophore functional group that absorbs energy of a specific wavelength and re-emits energy at a different (but equally specific) wavelength. Fluorophores include, but are not limited to fluorescein, rhodamine, quantum dots, and green fluorescent protein. A large number of fluorophores are available from various commercial sources (e.g., www.invitrogen.com/site/us/en/home/brands/Molecular-Probes.html?).

As used herein, a “hapten” is understood as a small molecule that can be specifically bound by an antibody. Typically a hapten can only elicit an immune response only when attached to a large carrier such as a protein; the carrier may be one that also does not elicit an immune response by itself. Haptens include, but are not limited to DiethyleneTriaminePentaacetic Acid (DTPA), aniline and its carboxyl derivatives (o-, m-, and p-aminobenzoic acid); fluorescein, biotin, digoxigenin, dinitrophenol, and polyethylene glycol (PEG).

The term “monoclonal antibody” or “mAb” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigen. Furthermore, in contrast to polyclonal antibody preparations that typically include different antibodies directed against different determinants (epitopes), each mAb is directed against a single determinant on the antigen. The modifier “monoclonal” is not to be construed as requiring production of the antibody by any particular method or that the monoclonal antibody is includes a full complement of six CDRs as in a naturally occurring antibody.

As used herein, the terms “identify” or “select” refer to a choice in preference to another. In other words, to identify a subject or select a subject is to pick out that particular subject from a group depending on characteristics of the subject, e.g., age, disease state, etc.

As used herein, “ligand” is understood any molecule, organic or biologic, natural or synthetic, which specifically binds to a binding site present on another molecule (e.g., antibody, receptor, etc). The interaction can be used to target the ligand to a particular site or molecule.

As used herein, “linked”, “operably linked”, “joined” and the like refer to a juxtaposition wherein the components described are attached to each other in a relationship permitting them to function in their intended manner. The components can be linked covalently (e.g., peptide bond, disulfide bond, non-natural chemical linkage), through hydrogen bonding (e.g., knob-into-holes pairing of proteins, see, e.g., U.S. Pat. No. 5,582,996; Watson-Crick nucleotide pairing), or ionic binding (e.g., chelator and metal) either directly or through linkers (e.g., peptide sequences, typically short peptide sequences; nucleic acid sequences; or chemical linkers). Linkers can be used to provide separation between active molecules so that the activity of the molecules is not substantially inhibited (less than 10%, less than 20%, less than 30%, less than 40%, less than 50%) by linking the first molecule to the second molecule. Linkers can be used, for example, in joining binding sites to each other and/or joining payload molecules to tethers. As used herein, molecules that are linked, but no covalently joined, have a binding affinity (Kd) of at least 10⁻³, 10⁻⁴, 10⁻⁵, 10⁻⁶, 10⁻⁷, 10⁻⁸, 10⁻⁹, 10⁻¹⁰, 10⁻¹¹, or 10⁻¹², or any range bracketed by those values, for each other under conditions in which the reagents of the invention are used, i.e., typically physiological conditions.

As used herein, a “molar ratio” is understood as the relative number of one type of molecule to another, either in a mixture, or linked to each other. For example, if tether that is end labeled on one end with a tag, then the molar ratio of the tether to the tag is 1:1. If the tether is end labeled on both ends with a tag, then the molar ratio of the tether to the tag is 1:2.

A “molecule for detection” as used herein is any molecule that can be detected using the compositions provided herein by binding of the bispecific ligand to the molecule for detection either directly or indirectly.

As used herein, a “mixed payload polymer” is understood as a molecular tether to which at least two distinct, preferably at least three distinct (e.g., 2, 3, 4, 5, 6, 7, etc.) payload molecules are attached. In a preferred embodiment, the size and/or the arrangement of the payload molecules on the tether does not interfere with the binding of the bispecific ligand, either directly or indirectly, to the tether, and further having at least one of the following characteristics:

a) the payload molecules are sufficiently separated on the tether such that the activity of one payload molecule, e.g., enzymatic activity, therapeutic activity, binding activity, is not substantially inhibited by the tether or the other payload molecules on the tether as compared to a payload molecule not attached to a tether;

b) binding of a payload to a bispecific ligand does not substantially disrupt the activity of the remaining payload molecules on the tether as compared to a payload molecule in a mixed payload polymer not bound to a bispecific ligand, wherein, activity of the payload molecule is not reduced by more than 50%, more than 40%, more than 30%, more than 25%, more than 20%, more than 15%, more than 10%, as compared to the appropriate control;
c) each payload molecule is attached to the tether by a single linkage such that the payload molecules are not attached to each other except through the tether;

d) at least one of the distinct payload molecules has a molecular weight of at least 5 kDa, preferably at least 10 kDa, more preferably at least 15 kDa, at least 20 kDa, at least 25 kDa, at least 30 kDa, at least 40 kDa, at least 50 kDa, at least 60 kDa, or at least 75 kDa;

e) at least one of the payload molecules is PEG.

In addition to having at least one of the characteristics set forth in a), b), c), d), or e) in certain embodiments, in a mixed payload polymer, the payload molecules on the tether are arranged to prevent the payload molecules from interfering with each other, and prevent the tether and any other molecules present on the tether from interfering with the activity of the payload.

The term “polynucleotide” means a polymeric form of two or more nucleotides, either ribonucleotides or deoxynucleotides, modified form of either type of nucleotide (e.g., PNA, LNA, phosphorothioate backbone, modified sugar, modified base), and chimeric ribonucleotides or deoxynucleotides that include mixtures of naturally and/or non-naturally occurring nucleotides. The term includes single and double stranded forms of nucleic acids.

The term “isolated polynucleotide” shall mean a polynucleotide (e.g., of genomic, cDNA, or synthetic origin, or some combination thereof) that, by virtue of its origin, the “isolated polynucleotide” is not associated with all or a portion of a polynucleotide with which the “isolated polynucleotide” is found in nature; is operably linked to a polynucleotide that it is not linked to in nature; or does not occur in nature as part of a larger sequence.

As used herein, the term “obtaining” is understood herein as manufacturing, purchasing, or otherwise coming into possession of.

“Payload” as used herein is any molecule attached to a tether than binds a first site on a bispecific ligand, and is preferably not present in the sample to which the second binding site on the bispecific ligand binds. An “active payload” as used herein refers to any molecule, or a set of molecules, which serves a detection, diagnostic or therapeutic function, including, but not limited to, detectable labels including diagnostic agents, and therapeutic agents, typically for delivery to a target site. In certain embodiments, the active payload molecules are the same, e.g., all a specific enzyme, fluorophore, therapeutic agent, biotin. In certain embodiments, the payload molecules are a mixture of molecules wherein essentially all of the molecules attached to the payload, e.g., 80%, 85%, 90%, 95%, 96%, 97%, 98%, 99%, 99.5%, 99.9%, or more have a diagnostic or a therapeutic function when not attached to the tether. Therefore, as used herein, a payload molecule does not include a molecule used to change the charge or solubility of the tether that does not, independent of the mixed payload polymer, have a detection, therapeutic, or diagnostic function, e.g., succinylate residues. In certain embodiments, the payload molecules are all therapeutic agents. In certain embodiments, the payload molecules are all diagnostic agents. In certain embodiments, the payload molecules are all in vivo diagnostic agents. In certain embodiments, the payload molecules are all therapeutic agents or in vivo diagnostic agents. “Distinct” payload molecules as used herein are molecules that can be distinguished using the bispecific ligands of the invention and are typically chemically distinct entities. However, it is understood that two PEG molecules may not be chemically identical, but can be recognized by the same anti-PEG antibody. Thus, two such PEG molecules that could be recognized by the same anti-PEG antibody would not be considered to be distinct payload molecules, although not chemically identical.

As used herein, “pharmaceutically acceptable” is understood as being of a quality acceptable for administration to a subject, preferably a human subject. Pharmaceutically acceptable compounds have a high level of purity, e.g., at least 95%, 96%, 97%, 98%, 99%, 99.5%, 99.9% or more pure, and are typically stored under conditions to maintain the quality of the product. Each component of a composition for administration to a human subject, and preferably for administration to a non-human subject, should be pharmaceutically acceptable. Similarly, reagents used for diagnostic methods, particularly diagnostics in relation to human subjects should have a similarly high level of purity, but need not be safe for ingestion by a human.

“Polymer” as used herein any molecule composed of repeating structural units (monomers) typically connected by covalent chemical bonds. Examples of polymers include, even when not explicitly specified and without limitation: polylysine (PL), polyglutamic acid (PGA), polyaspartic acid (PAA), lysine/glutamic acid copolymer (PL/GA), etc. Polymers include polycation polymers, such as, without limitation, poly(allylamine), poly(dimethyldiallyammonim chloride) polylysine, poly(ethylenimine), poly(allylamine), and natural polycations such as dextran amine, polyarginine, chitosan, gelatine A, and/or protamine sulfate. In other instances, polyanion polymers are used, including, without limitation, poly(styrenesulfonate), polyglutamic or alginic acids, poly(acrylic acid), poly(aspartic acid), poly(glutaric acid), and natural polyelectrolytes with similar ionized groups such as dextran sulfate, carboxymethyl cellulose, hyaluronic acid, sodium alginate, gelatine B, chondroitin sulfate, and/or heparin. Further polymers can include, without limitation, substantially pure carbon lattices (e.g., graphite), dextran, polysaccharides, polypeptides, polynucleotides, acrylate gels, polyanhydride, poly(lactide-co-glycolide), polytetrafluoroethylene, polyhydroxyalkonates, cross-linked alginates, gelatin, collagen, cross-linked collagen, collagen derivatives (such as succinylated collagen or methylated collagen), cross-linked hyaluronic acid, chitosan, chitosan derivatives (such as methylpyrrolidone-chitosan), cellulose and cellulose derivatives (such as cellulose acetate or carboxymethyl cellulose), dextran derivatives (such carboxymethyl dextran), starch and derivatives of starch (such as hydroxyethyl starch), other glycosaminoglycans and their derivatives, other polyanionic polysaccharides or their derivatives, polylactic acid (PLA), polyglycolic acid (PGA), a copolymer of a polylactic acid and a polyglycolic acid (PLGA), lactides, glycolides, and other polyesters, polyglycolide homopolymers, polyoxanones and polyoxalates, copolymer of poly(bis(p-carboxyphenoxy)propane)anhydride (PCPP) and sebacic acid, poly(l-glutamic acid), poly(d-glutamic acid), polyacrylic acid, poly(dl-glutamic acid), poly(l-aspartic acid), poly(d-aspartic acid), poly(dl-aspartic acid), polyethylene glycol, copolymers of the above listed polyamino acids with polyethylene glycol, polypeptides, such as, collagen-like, silk-like, and silk-elastin-like proteins, polycaprolactone, poly(alkylene succinates), poly(hydroxy butyrate) (PHB), polybutylene diglycolate), nylon-2/nylon-6-copolyamides, polydihydropyrans, polyphosphazenes, poly(ortho ester), poly(cyano acrylates), polyvinylpyrrolidone, polyvinylalcohol, poly casein, keratin, myosin, and fibrin, silicone rubbers, or polyurethanes, and the like. Other biodegradable materials that can be used include naturally derived polymers, such as acacia, gelatin, dextrans, albumins, alginates/starch, and the like; or synthetic polymers, whether hydrophilic or hydrophobic. These polymers can be synthesized using known methods, isolated from natural sources, or, in some cases, commercially obtained. In certain instances, biodegradable and/or biocompatible polymers are used.

The term “polypeptide,” as used herein, refers to any polymeric chain of amino acids. The terms “peptide” and “protein” are used interchangeably with the term polypeptide and also refer to a polymeric chain of amino acids. The term “polypeptide” encompasses native or artificial proteins, protein fragments, and polypeptide analogs of a protein sequence. A polypeptide may be monomeric or polymeric. Use of “polypeptide” herein is intended to encompass polypeptides, and fragments and variants (including fragments of variants) thereof, unless otherwise stated.

The term “isolated protein” or “isolated polypeptide” is a protein or polypeptide that by virtue of its origin or source of derivation is not associated with naturally associated components that accompany it in its native state; is substantially free of other proteins from the same species; is expressed by a cell from a different species; or does not occur in nature. Thus, a polypeptide that is chemically synthesized or synthesized in a cellular system different from the cell from which it naturally originates will be “isolated” from its naturally associated components. A protein may also be rendered substantially free of naturally associated components by isolation, using protein purification techniques well known in the art. For example, a protein may be 90% pure, 95% pure, 97% pure, 98% pure, 99% pure, or more, that is free of other components naturally occurring with the protein or nucleic acid, as determined by routine methods in the art.

A “population” of molecules as used herein is understood as a group of molecules (e.g., more than two) of a specific type, such as those provided herein, whose characteristics are defined based on the group of molecules as a whole. For example, a population can be defined by the characteristics of the population on average, e.g., a population of tethers may have about 1.5, 2, 2.5, 3, 3.5, 4, 4.5, 5, etc. payload molecules per tether. Alternatively, a population can be defined by a characteristic shared by a defined percent of the population, e.g., a population of tethers in which at least 70%, 80%, 90%, 95%, or more that have 6 or fewer, 5 or fewer, 4 or fewer, 3 or fewer, or 2 payload molecules. Similarly, populations of bispecific ligands can include populations of at least 70%, 80%, 90%, 95%, or more that have only two binding sites; or that have more than two binding sites.

As used herein, “qualitative” detection is understood as performing an assay to determine if a target is present in a sample in an amount greater than or less than the detection limit of the method. “Semi-quantitative detection” is understood as performing an assay to determine the amount of a target in a sample yielding an approximation of the quantity or amount of a substance; falling short of a quantitative result, e.g., grading the amount of target present as below the limit of detection, low, medium, or high; analysis using subjective rather than objective measures. “Quantitative detection” is understood as performing an assay for detection of a target in which numeric values as a result of subjective analysis. It is understood that quantitative detection can provide outcomes within a numerical range, e.g., a value +/−a standard deviation or error.

As used herein, “radioactive label”, “radiolabel”, or “radionuclide” as a detectable label includes, but is not limited to iodine (¹³¹I or ¹²⁵I), yttrium (⁹⁰Y), lutetium (¹⁷⁷Lu), actinium (²²⁵Ac), praseodymium (¹⁴²Pr or ¹⁴³Pr), astatine (²¹¹At), rhenium (¹⁸⁶Re or ¹⁸⁷Re), bismuth (²¹²Bi or ²¹³Bi), indium (¹¹¹In), technetium (⁹⁹mTc), phosphorus (³²P), rhodium (¹⁸⁸Rh), sulfur (³⁵S), carbon (¹⁴C), tritium (³H), chromium (⁵¹Cr), chlorine (³⁶Cl), cobalt (⁵⁷Co or ⁵⁸Co), iron (⁵⁹Fe), selenium (⁷⁵Se), or gallium (⁶⁷Ga). Many radioactive labels are used as in vivo diagnostic agents.

The term “sample” as used herein refers to any material in which a target molecule can be detected using the methods provided herein. Typically a sample is a biological sample such as a collection of similar fluids, cells, or tissues isolated from a subject (e.g., by surgical resection, biopsy, autopsy/necropsy, stored historical samples) or cell culture. The term “sample” includes any body fluid (e.g., urine, serum, blood fluids, lymph, gynecological fluids, cystic fluid, ascetic fluid, ocular fluids and fluids collected by bronchial lavage and/or peritoneal rinsing), ascites, tissue samples or a cell from a subject. Other subject samples include tear drops, serum, cerebrospinal fluid, feces, sputum, and cell extracts. Subject samples also include tissue sections, e.g., sections prepared for microscopy for diagnostic or research purposes. Samples can also include environmental samples. As used herein, the sample can be in the subject and detected using imaging methods.

A “secondary antibody” or “SAB” is understood as an antibody that detects another antibody (i.e., a primary antibody) based on the species of the primary antibody and typically the immunoglobulin type. Secondary antibodies are usually anti-Mouse (αMs), anti-Rabbit (αRb), anti-Goat (αGt), anti-Human (αHu), etc and are further specific to IgG, IgM, IgA, IgE, etc. Secondary antibodies can include a detectable label and are commercially available (e.g., www.piercenet.com/browse.cfm?fldID=010401).

As used herein, “small molecule” is understood as compound having a molecular weight of less than about 2000 Da, 1500 Da, 1250 Da, 1000 Da, 750 Da, or 500 Da. In certain embodiments, the small molecule is not a nucleic acid. In certain embodiments, the small molecule is not a peptide. In certain embodiments, the small molecule is an organic compound. In certain embodiments, the small molecule is an inorganic compound.

As used herein, “solid support” is understood as any macroscopic solid material to which a target for detection using the methods of the invention can be attached, e.g., a tissue culture or ELISA plate, a slide, membranes, polymer beads, or nanoparticles that may be coated with a capture reagent, e.g., antibody, cell, extracellular matrix component(s), adhesive, etc. Samples on solid supports can be used to perform diagnostic assays including ELISA assays, immunohistochemical assays, and lateral flow assays.

“Specific” and “specificity” in the context of an interaction between members of a specific binding pair (e.g., a ligand and a binding site, an antibody and an antigen, biotin and avidin) refer to the selective reactivity of the interaction. The phrase “specifically binds to” and analogous phrases refer to the ability of antibodies (or antigenically reactive fragments thereof) to bind specifically to an antigen (or a fragment thereof) and not bind specifically to other entities. Specific binding is understood as a preference for binding a certain antigen, epitope, receptor ligand, or binding partner with at least a 10³, 10⁴, 10⁵, 10⁶, 10⁷, 10⁸, 10⁹-fold preference over a control non-specific antigen, epitope, receptor ligand, or binding partner. It is understood that various proteins can share common epitopes or other binding sites (e.g., kinase reactive sites). In certain embodiments, binding sites may bind more than one ligand, but still can be considered to have specificity based on binding preference as compared to a non-specific antigen and/or by having certain binding kinetic parameters. Methods of selecting appropriate non-specific controls are within the ability of those of skill in the art.

The terms “specific binding” or “specifically binding,” as used herein, in reference to the interaction of an antibody, a protein, or a peptide with a second chemical species, mean that the interaction is dependent upon the presence of a particular structure (e.g., an antigenic determinant or epitope) on the chemical species; for example, an antibody recognizes and binds to a specific protein structure, rather than to proteins generally. If an antibody is specific for epitope “A,” the presence of a molecule containing epitope A (or free, unlabeled A) in a reaction containing labeled “A” and the antibody will reduce the amount of labeled A bound to the antibody.

“Specific binding” as used herein can also refer to binding pairs based on binding kinetics such as Kₒₙ, K_{off}, and K_{D}. For example, ligand can be understood to bind specifically to its target site if it has a K_{off} of 10⁻² sec⁻¹ or less, 10⁻³ sec⁻¹ or less, 10⁻⁴ sec⁻¹ or less, 10⁻⁵ sec⁻¹ or less, or 10⁻⁶ sec⁻¹ or less; and/or a K_{D} of 10⁻⁶ M or less, 10⁻⁷ M or less, 10⁻⁸M or less, 10⁻⁹ M or less, 10⁻¹⁰ M or less, or 10⁻¹¹ M or less, or 10⁻¹²M or less. Binding assays are typically performed under physiological conditions.

“Specific binding partner” is a member of a specific binding pair. A specific binding pair comprises two different molecules, which specifically bind to each other through chemical or physical means. Therefore, in addition to antigen and antibody specific binding pairs of common immunoassays, other specific binding pairs can include biotin and avidin (or streptavidin), carbohydrates and lectins, complementary nucleotide sequences, effector and receptor molecules (e.g., acetylcholine and muscarinic receptor), cofactors and enzymes, enzyme inhibitors and enzymes, chelators and heavy metals, cytokines and receptors, drug-drug receptors, e.g., dopamine and dopamine receptor, hormone or hormone analog and hormone receptor (e.g., estrogen or estradiol and estrogen receptor); and the like. Furthermore, specific binding pairs can include members that are analogs of the original specific binding members, for example, an analyte-analog. Immunoreactive specific binding members include antigens, antigen fragments, and antibodies, including monoclonal and polyclonal antibodies as well as complexes, fragments, and variants (including fragments of variants) thereof, whether isolated or recombinantly produced.

As used herein, the term “subject” refers to human and non-human animals, including veterinary subjects. The term “non-human animal” includes all vertebrates, e.g., mammals and non-mammals, such as non-human primates, mice, rabbits, sheep, dog, cat, horse, cow, chickens, amphibians, and reptiles. In a preferred embodiment, the subject is a human and may be referred to as a patient.

“Target” as used herein is understood as any molecule, organic or biologic, natural or synthetic, of which the presence or concentration can be detected using the compositions and methods provided herein. A target includes, but is not limited to an antigen; a receptor (for example expressed on a cell surface); or a nucleic acid sequence (for example DNA, RNA, microRNA, or any other genetic material). A target can include a molecule endogenous to a sample, e.g., a protein in or on the surface of a cell, or can be a molecule not endogenous to the sample, e.g., a primary antibody that binds to the protein endogenous to the sample. As used herein, the target is one of the molecules bound directly by the bispecific ligand.

As used herein, a “tether” is understood as a molecule to which at least two of each of two distinct payload molecules can be attached. In certain embodiments, the tether is a polymer. In certain embodiments, the tether further includes an analytical tag. In certain embodiments, the tether is linear (i.e., unbranched, has only two ends). In certain embodiments, the tether is branched (i.e., has more than two ends). In certain embodiments, the tether is negatively charged. In certain embodiments, the tether is present in a molecule that consists essentially of the tether, at least two of each of two distinct payload molecules, and an analytical tag. In certain embodiments, the tether containing molecule further comprises a capturing tag. In certain embodiments, the tether is joined to DTPA. In certain embodiments, the tether is not joined to DTPA. In certain embodiments, the tether is homogenously modified to alter the properties of the tether, e.g., decrease positive charge/increase negatively charge of the polymer, modify the solubility of the polymer, blocking reactive sites on the tether. Such groups used for modification of the general properties of tether are not payload molecules. In certain embodiments, the tether is not succinylated. In certain embodiments, the tether includes one or more biotin moieties that may or may not be payload molecules.

As used herein, a “therapeutic agent” is understood a molecule, organic or biologic, natural or synthetic, or a radioisotope which exerts a therapeutic effect on its intended target. A therapeutic agent may be, without limitation any pharmaceutical used for chemotherapy, any radiopharmaceutical used for radiation therapy, or other, including, but not limited to, in certain embodiments, the chemotherapeutic agent is selected from the group consisting of doxorubicin (DOXO), 6-mercaptopurine, 6-thioguanine, cytarabine, 5-fluorouracil decarbazine, mechlorethamine, thioepa chlorambucil, CC-1065, melphalan, carmustine (BSNU), lomustine (CCNU), cyclothosphamide, busulfan, dibromomannitol, streptozotocin, mitomycin, cis-dichlorodiamine platinum (II) (DDP) cisplatin, daunorubicin, paclitaxel, dactinomycin, bleomycin, mithramycin, anthramycin (AMC), vincristine, vinblastine, taxol, maytansinoids, cytochalasin B, gramicidin D, ethidium bromide, emetine, etoposide, tenoposide, colchicin, dihydroxy anthracin dione, mitoxantrone, mithramycin, 1-dehydrotestosterone, glucocorticoids, procaine, tetracaine, lidocaine, propranolol, puromycin, and calicheamicin.

The articles “a”, “an”, and “the” are used herein to refer to one or to more than one (i.e. to at least one) of the grammatical object of the article unless otherwise clear from context. By way of example, “an element” means one element or more than one element. Similarly, unless otherwise clear from context, “the” is similarly understood to be either singular or plural.

The term “including” is used herein to mean, and is used interchangeably with, the phrase “including but not limited to”.

The term “or” is used herein to mean, and is used interchangeably with, the term “and/or,” unless context clearly indicates otherwise.

The term “such as” is used herein to mean, and is used interchangeably, with the phrase “such as but not limited to”.

The transitional term “comprising”, which is synonymous with “including”, “containing”, or “characterized by”, is inclusive or open-ended and does not exclude additional, unrecited elements or method steps.

The transitional phrase “consisting essentially of” is understood to have the meaning provided by US patent law and limits the scope of a claim to the specified materials or steps and those that do not materially affect the basic and novel characteristic(s) of the claimed invention. As used herein, a pharmaceutical composition or therapeutic agent that “consists essentially of” a tether, a payload, and an analytical tag, can include other components that do not materially affect the basic and novel characteristics of the compound, e.g., do not include other ligands for binding by antibodies, do not include further detectable labels. When the phrase “consists essentially of” appears in a clause of the body of a claim, rather than immediately following the preamble, it limits only the element set forth in that clause; other elements that may materially affect the characteristics of the claimed material or method are not excluded from the claim as a whole.

The transitional phrase “consisting of” excludes any element, step, or ingredient not specified in the claim. “Consisting of” defined as “closing the claim to the inclusion of materials other than those recited except for impurities ordinarily associated therewith. It is understood that the presence of a non-related component, e.g., a bottle, blister pack, vial, tube, or bag, to contain a pharmaceutical composition falls within the scope of a pharmaceutical composition comprising the specified components. A claim which depends from a claim which “consists of” the recited elements or steps cannot add an element or step. When the phrase “consists of” appears in a clause of the body of a claim, rather than immediately following the preamble, it limits only the element set forth in that clause; other elements are not excluded from the claim as a whole.

Unless specifically stated or obvious from context, as used herein, the term “about” is understood as within a range of normal tolerance in the art, for example within two standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein can be modified by the term about.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50, or fractional portions thereof, as appropriate.

The recitation of a listing of chemical group(s) in any definition of a variable herein includes definitions of that variable as any single group or combination of listed groups. The recitation of an embodiment for a variable or aspect herein includes that embodiment as any single embodiment or in combination with any other embodiments or portions thereof.

Terms of relative size as used herein are understood to have their usual meaning. For example, the “smallest payload” is understood as the payload molecule with the smallest (average) molecular weight. A “smaller payload” is understood as all payload molecules other than the payload with the highest (average) molecular weight. Similarly, the “largest payload” has the highest average molecular weight, and a “larger payload” is understood as all payload molecules other than the payload with the smallest (average) molecular weight.

Any compositions or methods provided herein can be combined with one or more of any of the other compositions and methods provided herein.

### DETAILED DESCRIPTION

Genomic sequencing and proteomics have advanced research for many clinical indications; however, a critical limitation for research is the enablement for identification and characterization of low abundance antigens or antibody-based biomarkers, e.g. auto-antibodies. Further, many monoclonal and polyclonal antibodies are now available for use in immunoassays, immunohistochemistry (IHC) and test systems where results and interpretations are limited by the inability to accurately detect low abundance antigen-antibody interactions. The invention provides ultrasensitive, robust and high-throughput methods and reagents for use in those methods that enable the rapid and ultrasensitive detection of biomarkers. This technology overcomes several existing technical limitations for biomarker detection, including assay sensitivity and detectability, assay run time and utility. The compositions and methods provided herein allow detection down to the zeptomole range (10⁻²¹ moles) (e.g., down to 10⁻²¹ moles, down to 10⁻²⁰ moles, down to 10⁻¹⁹ moles, down to 10⁻¹⁸ moles, down to 10⁻¹⁷ moles, down to 10⁻¹⁶ moles, down to 10⁻¹⁵ moles, down to 10⁻¹⁴ moles, or down to 10⁻¹³ moles). This detection limit translates to measurements in the attogram (10⁻¹⁸ g/ml, e.g., 1×10⁻¹⁸ g/ml, 10×10⁻¹⁸ g/ml, 100×10⁻¹⁸ g/ml) or femtogram/ml (1×10⁻¹⁵ g/ml, 10×10⁻¹⁵ g/ml, 100×10⁻¹⁵ g/ml) range, which surpasses current detection limits in the picogram/ml (10⁻¹² g/ml) range. This enhancement is achieved by greatly amplifying signal intensity while maintaining excellent signal to noise ratios as demonstrated in the Examples.

The invention provides mixed polymer payload molecules and their use in the delivery of payloads, e.g., detection reagents and therapeutic reagents, to a specific site using a bispecific ligand. The invention is based at least in part, on the demonstration herein that a payload on a tether is more effectively captured at its target site when the payload being captured is a larger payload. Specifically, as shown in the examples below, a tether containing both horse radish peroxidase (HRP) (molecular weight about 44 kDa) and DTPA (molecular weight 393 Da) was bound with more efficiency by antibodies specific to HRP than by antibodies specific to DTPA. This increase in signal is not accompanied by an increase in background signal. Therefore, the reagents and methods provide greater sensitivity than prior detection methods.

In certain embodiments, the mixed payload polymers provided herein include the use of relatively large haptens, e.g., PEG, to increase the delivery of relatively small active payloads, e.g., detectable label, therapeutic agents, or diagnostic agents. For example, in preferred embodiments, the hapten is at least 5-fold, 6-fold, 7-fold, 8-fold, 9-fold, 10-fold, 11-fold, 12-fold, 13-fold, 14-fold, or 15-fold larger than the active payload. In certain embodiments, the hapten has an average molecular weight of at least about 2 kDa, 3, kDa, 4 kDa, preferably at least about 5 kDa, more preferably at least 10 kDa, even more preferably at least 15 kDa, at least 20 kDa, at least 25 kDa, at least 30 kDa, at least 40 kDa, at least 50 kDa, at least 60 kDa, or at least 75 kDa. The use of a hapten that is larger than the active payload, or at least one active payload on the tether, allows for increased delivery of the active payload to the target site. In certain embodiments, the hapten that is larger than at least one of the active payloads is PEG. In certain embodiments, the use of a larger hapten payload than the active payload provides a method to increase the delivery of a small active payload (e.g., less than 2 kDa, less than 1.5 kDa, less than 1 kDa, less than 750 Da, less than 500 Da) to a target site by delivering the small active payload in a mixed payload polymer containing a relatively large hapten for binding to a bispecific ligand. In certain embodiments, the delivery of the relatively small active payload is increased by at least 50%, 75%, 100%, 150%, 200%, 250%, 300%, or 500%, as compared to capturing the small payload directly. In certain embodiments, the mixed payload polymer includes multiple active payload agents that are smaller than the larger hapten.

In certain embodiments, the mixed payload polymer can further include at least one additional active payload, e.g., an enzyme, e.g., HRP, alkaline phosphatase, luciferase, that is larger than the hapten, in addition to the active payload or payloads that are smaller than the hapten.

The invention provides methods for use of such mixed payload polymers wherein a larger payload is captured by a bispecific ligand to target an active payload to a desired site.

The mixed payload polymers of the invention can be used for methods of delivery of two or more distinct active payloads to a target site by the use of a single bispecific ligand. For example, a bispecific ligand that binds to a protein of interest and PEG can be used for detection of the protein of interest in both an ELISA or western blot assay as well as an immunofluorescence or FACS assay using a mixed polymer peptide containing a fluorophore that is smaller than the PEG hapten, a PEG hapten, and an HRP that is larger than the hapten.

In certain embodiments, an inactive hapten for capture has no activity directly for detection outside of the context of the mixed peptide polymer. In certain embodiments, the inactive hapten is larger than all of the payload molecules.

In certain embodiments, the polymer, all of the payload types are distributed about evenly and homogeneously throughout the length of the polymer. That is, the polymer is synthesized without a bias as to the location of the payloads on the polymer.

In certain embodiments, each of the active payload molecules is present in about the same molar amount as the hapten. In certain embodiments, the active payload is present in a molar excess to the hapten. In certain embodiments, the total number of active payload molecules are present in at least a 2-fold, 3-fold, 4-fold, 5-fold, 10-fold, 20-fold, 30-fold, 40-fold, 50-fold, 75-fold, 100-fold, 200-fold, 300-fold, 400-fold, 500-fold, 750-fold, 1000-fold excess as compared to the hapten. In certain embodiments, the tether is labeled on one or both ends with the hapten.

In certain embodiments, when the mixed payload polymer contains multiple active payloads, the payloads are present in about equimolar amounts to each other. In certain embodiments, the amount of each payload on the tether is selected independently of the amount of the other payload(s) present on the tether. In certain embodiments, one of the active payloads is present at least a 1.5-fold, 2-fold, 3-fold, 4-fold, 5-fold, 6-fold, 7-fold, 8-fold, 9-fold, 10-fold, 20-fold, 30-fold, 40-fold, 50-fold, 75-fold, 100-fold excess of the other active payload.

The ultrasensitive detection methods provided herein can enhance disease diagnosis and management, including for cancer, transplantation, infectious and neurological diseases. For almost every disease, earlier diagnosis and more accurate measurement of disease progression allow for earlier and less expensive medical intervention and better treatment outcomes. Further, the ability to probe tissue samples for low abundance antibody-antigen interactions also fundamentally expands the research findings possible in both preclinical and clinical research arenas. The ability to amplify detection in smaller, e.g. droplet, quantities of sample can also accelerate the implementation of Point of Care (POC) antibody-based monitoring for primary care physicians and personalized medicine applications, exemplified by existing lateral flow immunoassay based methodologies. Additional potential benefits of enhanced sensitivity and detectability include resource and cost savings from: (1) shorter assay times; (2) sample volume reductions for sample conservation and decreased reagent use; (3) elimination of matrix effects by dilution; and (4) utility for multiple clinical and preclinical application formats, including high-throughput screening, ELISA, chemiluminescence, western blot and immunohistochemistry.

The methods provided herein include the use of a bispecific ligand with one binding site having high specificity for the target while the other end has high affinity for a mixed payload polymer, i.e., a tether molecule linked to functional payload molecules which include, for example, detectable labels and/or therapeutic agents, and optionally a hapten that is not the smallest molecule attached to the tether.

Further, in certain embodiments, bispecific ligands that have only two binding sites are preferred. Various methods of generating bispecific ligands can result in more than two binding sites being present. Synthetic methods can be selected, e.g. recombinant expression, purification methods, to select bispecific ligands that have only one binding site for the target and one binding site for the payload.

Moreover, as the bispecific ligand and mixed payload polymer are not covalently bound, a pre-targeting approach can be used, e.g., for in vivo imaging applications, which further reduces background. Also, the methods provided herein include direct modification of the ligand that binds to the target in the sample, e.g., the primary antibody for use in a direct assay thereby enhancing maximal sensitivity by reducing non-specific binding and eliminating opportunity for errors and additional non-specific binding in the multiple steps of a typical indirect assay. The methods provide herein result in both greater signal amplification and higher signal-to-noise ratios, resulting in a significant sensitivity increase as compared to presently available methods.

The invention provides reagents and methods for detection of targets in samples and subjects. The method relies on the use of bispecific ligands, wherein one of the binding sites binds to a target in the sample or subject, and the other site in the bispecific ligand binds to a tether that contains at least two distinct payload molecules. The payload molecule is an active agent, e.g., a detectable label, a drug, for delivery to the target site. In certain embodiments, the payload may be a mixture of two or more active agents. In a preferred embodiment, the bispecific ligand binds directly to the payload.

In prior methods, polymers linked to payload molecules were further modified to include low molecular weigh non-payload antigens that do not have a diagnostic or therapeutic function independent of being a component of the tether, for binding to the bispecific ligands to target the payload to the target site. As demonstrated herein, the amount of payload that is delivered to a target site is substantially increased when the bispecific ligand binds to a larger payload on the tether. This is particularly true when the payload is substantially larger than the non-payload antigen on the tether, e.g., at least 2 times as large, at least 3 times as large, at least 4 times as large, at least 5 times as large, at least 7 times as large, at least 10 times as large, at least 15 times as large, at least 20 times as large, at least 25 times as large, at least 30 times as large, at least 40 times as large, or at least 50 times as large, as determined by molecular weight. For example, when the payload is an enzyme, e.g. horse radish peroxidase or alkaline phosphatase, and the non-payload antigen is DTPA. As demonstrated herein, the sensitivity of detection can be increased by at least 0.5-fold, 1-fold, 2-fold, 3-fold, 4-fold, 5-fold, 7-fold, 10-fold, 15-fold, 20-fold, 25-fold, 30-fold, or more (or any range bracketed by any of the values) by capturing a larger payload rather than a smaller payload attached to the tether. Molecules present on the tether to change the charge, solubility, or other physical properties of the tether that do not have therapeutic or diagnostic function in the absence of the tether and/or that are not captured by a bispecific ligand are not payload molecules.

The invention further provides multimerized or “stacked” payload containing tethers. In certain embodiments, the payload containing tethers further include a biotin moiety that is not a payload molecule, and/or optionally an avidin moiety. The biotin-payload containing tethers are combined with an avidin moiety that is not a payload molecule, either free or attached to a tether, to form multimeric structures. By using biotin-avidin interactions for multimerization when neither biotin nor avidin is the payload, the activity of the payload molecules is not disrupted by the multimerization. By increasing the payload delivered to the site of interest, it is possible to further increase the signal amplification and therefore the sensitivity and/or the speed of the detection methods.

In certain embodiments, signal amplification is achieved by capturing a biotin-payload containing tether using the bispecific ligand, and subsequently contacting the biotin-payload containing tether with polymers having an avidin moiety and multiple biotin moieties which are subsequently detected using an appropriate reagent.

The compositions and methods provided herein further include methods for the detection of nucleic acids in samples using bispecific ligands of the invention. The use of the dual specific ligands including a nucleic acid as provided herein can be used for the detection of small amounts of nucleic acid without the need for amplification of the sequence, e.g., as in PCR, and without isolating the nucleic acid sequence from the sample, or separating the nucleic acid sequence of interest from other nucleic acid, e.g., as in blotting or chromatography methods. Therefore, the reagents and methods provide rapid detection of nucleic acids in samples with high sensitivity.

### In Vitro Detection

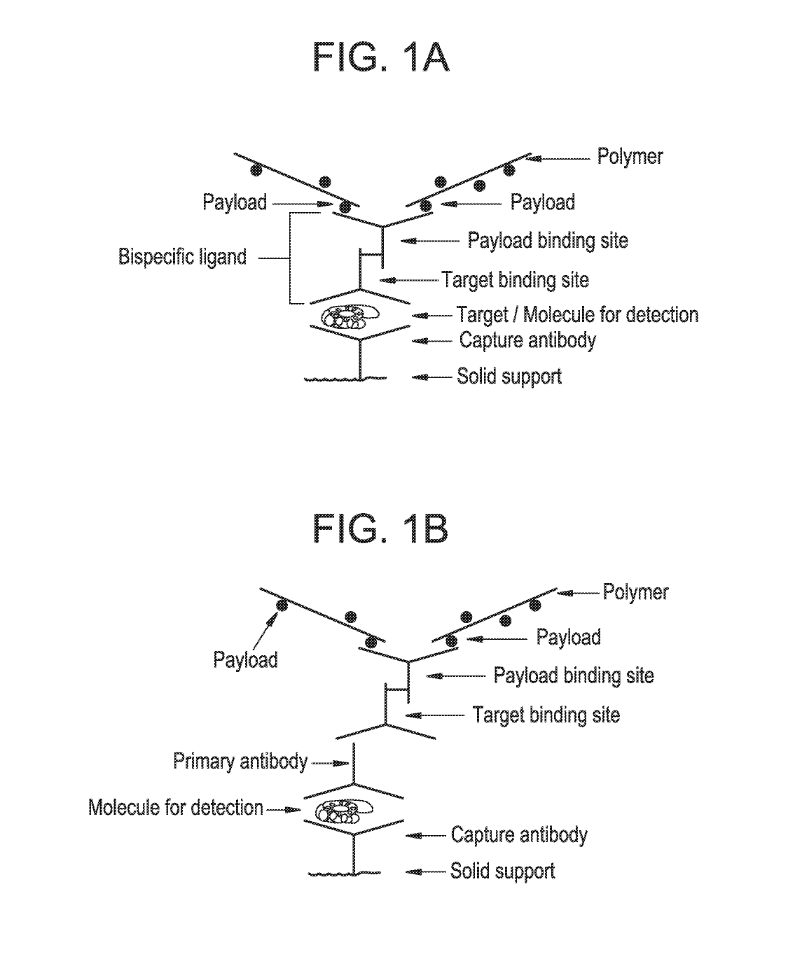

The compositions provide herein can be used for in vitro detection methods. For the sake of simplicity, the in vitro methods shown in FIGS. 1A-C are analogous to an ELISA type assay and are representative of how the reagents and methods provided herein can be used to assemble complexes for the detection of molecules attached to solid supports. However, it is understood that the target for detection could be attached using a different reagent by a different method to a different solid support, e.g., a slide for detection of a target in a tissue section or cell; or as a polypeptide array, to polymer beads for lateral flow assays, magnetic or non-magnetic microparticles, etc. Methods to prepare samples to expose intracellular antigens are well known. Similarly, selection of a detectable label appropriate for the in vitro method used is routine in the art.

FIGS. 1A-C schematically show various in vitro detection methods for use in combination with the payload containing tethers and bispecific ligands provided herein. For the sake of clarity, the schematics show a single type of payload molecule. Based on the teachings provided herein, it is clearly understood that multiple payloads, including active payloads and inactive hapten payloads of various sizes can be used. Further, it is well within the ability of those in the art to select specific controls, perform assays in replicates using serial dilutions, etc.

As shown in FIGS. 1A-C, a capture reagent in the form of a capture antibody is coated onto a solid support, e.g., an ELISA well. The well containing the capture antibody is contacted with the sample to capture the molecule for detection, e.g., an antigen. The well is washed to remove unbound sample and provide a target/molecule for detection attached to a solid support.

FIG. 1A shows an assembled complex in which the bispecific ligand binds directly to the molecule for detection and the payload attached to the polymer/tether. To assemble the complex in FIG. 1A, the prepared well to which the target/molecule for detection is bound is contacted with a bispecific ligand that includes a binding site for the antigen and a binding site for the payload of the payload containing polymer. The well is washed to remove the unbound bispecific ligand. The well is then contacted with the payload containing polymer under conditions to permit binding. The well is washed to remove unbound polymer.

FIG. 1B shows an assembled complex in which molecule for detection is bound by a primary antibody which, in turn, is bound by the bispecific ligand binds to the target/primary antibody and a payload on the polymer. Although a primary antibody is represented schematically in the figure, it is understood that any molecule that specifically binds the molecule for detection and be simultaneously bound by the bispecific ligand can be used. To assemble the complex in FIG. 1B, the prepared well to which the molecule for detection is bound is sequentially contacted with a primary antibody that specifically binds the molecule for detection, e.g., antigen, a wash solution, and a bispecific ligand that includes a binding site for the target/primary antibody and a binding site for the payload of the payload containing polymer. The well is washed to remove the unbound bispecific ligand. The well is then contacted with the payload containing polymer under conditions to permit binding. The well is washed to remove unbound polymer.

FIG. 1C shows an assembled complex in which molecule for detection is bound by a primary antibody which, in turn, is bound by a secondary antibody which, in turn, is bound by the bispecific ligand binds to the target/secondary antibody and the payload on the polymer. To assemble the complex in FIG. 1C, the prepared well to which the molecule for detection is bound is sequentially contacted with a primary antibody that specifically binds the antigen, a wash solution, a target/secondary antibody that binds the primary antibody, a wash solution, and a bispecific ligand that includes a binding site for the target/secondary antibody and a binding site for a payload of the payload containing polymer. Although a primary antibody and a secondary antibody are represented schematically in the figure, it is understood that any molecule that specifically binds the molecule for detection and can simultaneously be bound by the “secondary antibody” can be used as the “primary antibody”. Similarly, any molecule that can be used in the position schematically represented by the secondary antibody that can bind the “primary antibody” and the bispecific ligand specifically and simultaneously. In certain embodiments, the secondary antibody is a biotinylated antibody and the bispecific ligand includes a streptavidin moiety for binding the target/secondary antibody. The well is washed to remove the unbound bispecific ligand. The well is then contacted with the payload containing polymer under conditions to permit binding. The well is washed to remove unbound polymer.

The payload on the polymers in the assembled complexes shown schematically in FIGS. 1A-1C is detected using routine methods based on the specific payload used. For example, the payload may be a fluorescent label that is detected directly by exposing the complex to an appropriate wavelength of light. In another embodiment, the payload can be an enzyme that is detected calorimetrically or spectrophotometrically by contacting the payload with an appropriate enzyme substrate. If the payload is biotin, the biotin payload can be further contacted with a streptavidin-bound agents, e.g., containing enzyme or fluorophore for detection. Alternatively, when the payload is biotin, the signal may be further amplified prior to detection by contacting the payload with polymers containing at least one streptavidin moiety and multiple biotin moieties. The biotin moieties are subsequently contacted with streptavidin-bound agents for detection.

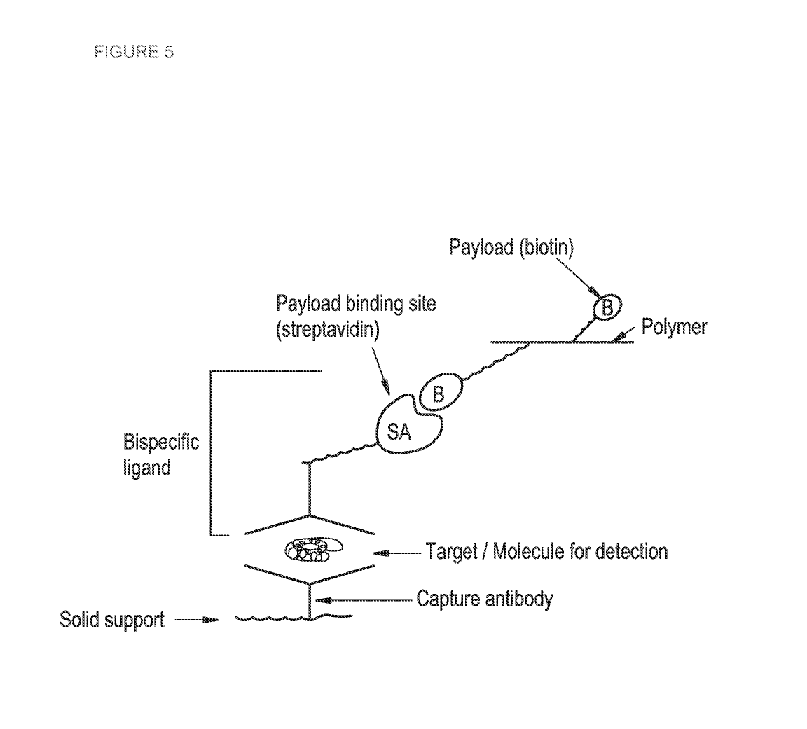

In certain embodiments, the method includes a primary or secondary antibody is attached to an avidin moiety and the mixed payload polymer includes biotin as the payload. In certain embodiments, the bispecific ligand includes an avidin moiety for binding to a biotin payload on a tether (see, e.g., FIG. 5).

Further, the reagents and methods can be readily modified for the detection of molecules other than antigens. In certain embodiments, cells expressing a cell surface receptor can be attached to the solid support and detected using a bispecific ligand that includes a specific receptor ligand (e.g., a hormone to bind a hormone receptor, a cytokine to bind a cytokine receptor, a drug to bind a drug receptor). In certain embodiments, the bispecific ligand can be contacted with the cells prior to fixing the cells.

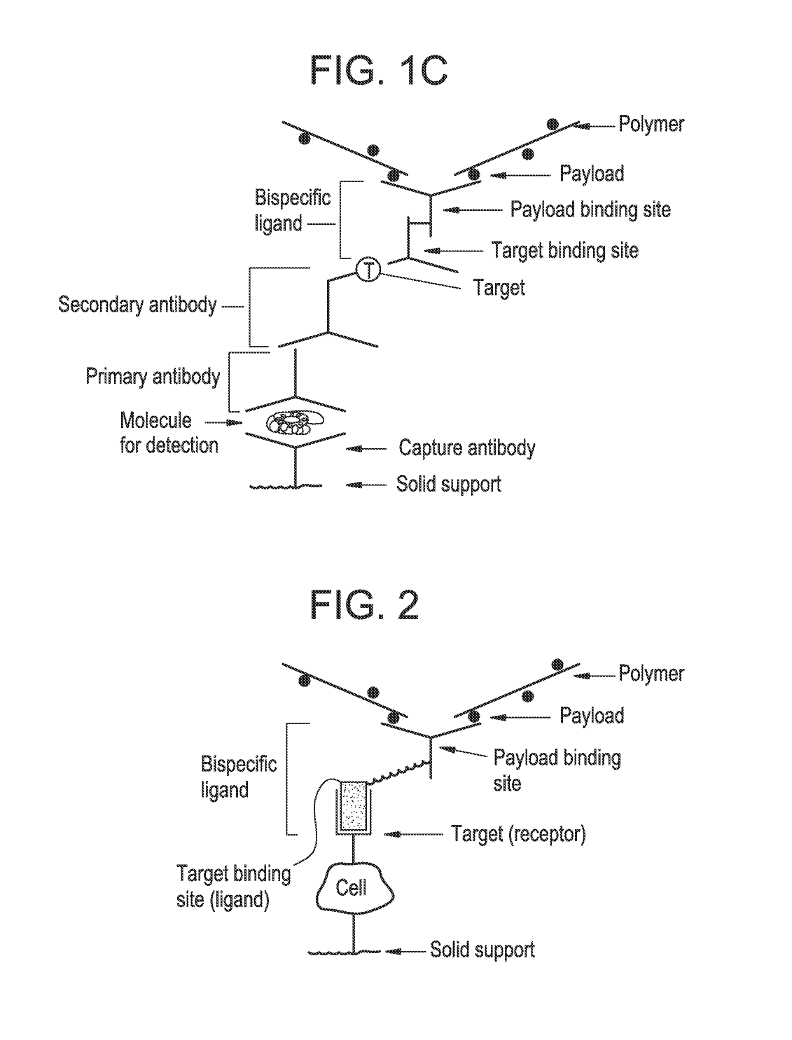

FIG. 2 is a schematic of a complex in which the receptor on the cell is the molecule for detection and the bispecific ligand shown includes a ligand for the receptor and an antibody for binding the payload. It is understood that the ligand could be conjugated a molecule other than an antibody, e.g., an avidin moiety, to bind a biotin payload. Further, it is understood that binding of the bispecific ligand directly to the molecule for detection is not required and that complexes similar to those shown in FIGS. 1B and 1C can be used with a receptor for detection using a ligand. Further, it is understood that a receptor does not need to be on a cell, but may be attached to the solid support by other methods.

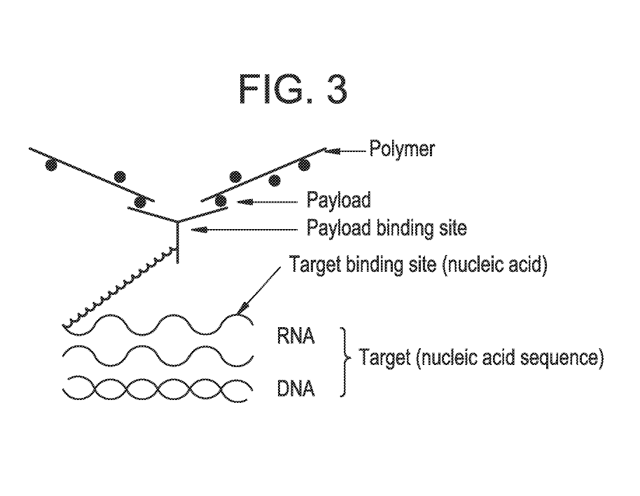

FIG. 3 is a schematic of an assembled complex in which the molecule for detection is a nucleic acid. Nucleic acids can be present, for example, in tissue samples or attached to solid supports by any method known in the art. Methods for denaturation of nucleic acids in samples and on supports are known in the art. As with the exemplary embodiments presented in FIGS. 1A-C and 2, the schematic shows one representation of the use of the method wherein the molecule for detection is a nucleic acid. It is understood that the nucleic acid could be conjugated a molecule other than an antibody, e.g., an avidin moiety, to bind a biotin payload. Further, it is understood that binding of the bispecific ligand directly to the molecule for detection is not required and that complexes similar to those shown in FIGS. 1B and 1C can be used with a nucleic acid for detecting a molecule in a sample. Further, it is understood that a nucleic acid can be used to detect another molecule by hybridization or by binding of the nucleic acid to a protein, e.g., a DNA binding protein.

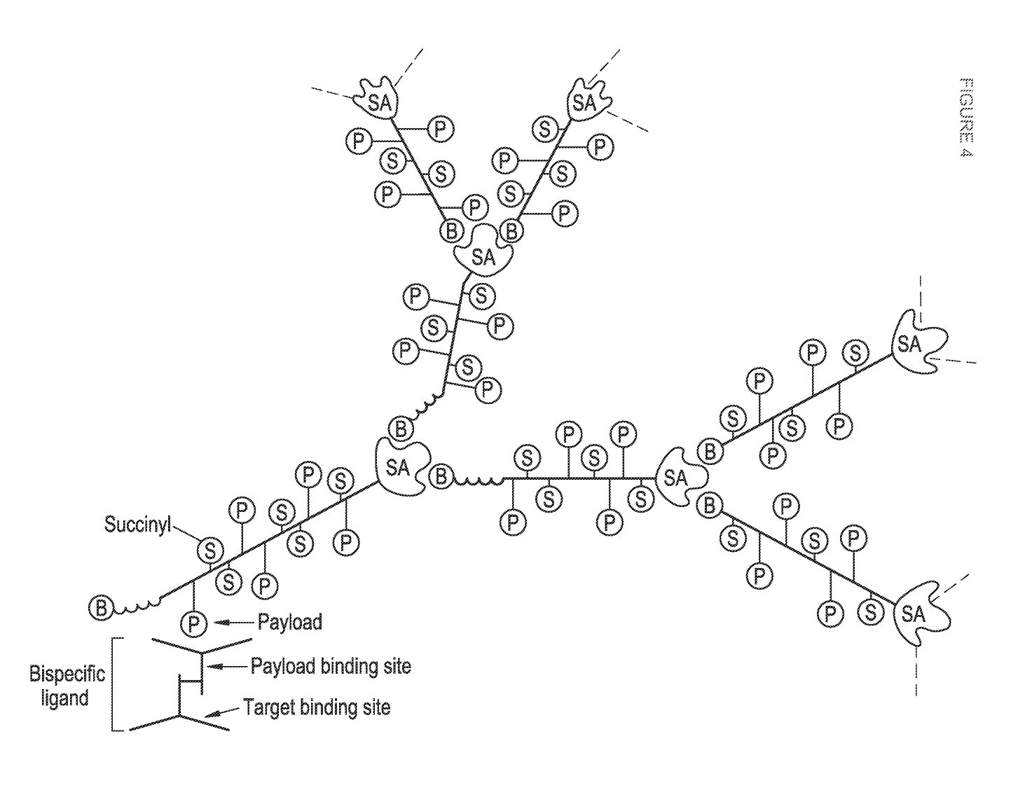

FIG. 4 shows a multimerized tethers in which the tethers have an avidin moiety, e.g., a streptavidin moiety, on one end and a biotin moiety on the other. Streptavidin has multiple biotin binding sites allowing for the formation of complex (i.e., other than linear) structures. Such multimerized tethers can be used to amplify the signal from the payload in the method. Tethers with different payloads can be multimerized into a single complex. Multimerized tethers do not need to be succinylated, as shown schematically in the figure.

### In Vivo Detection

A bispecific ligand including a ligand for binding to a cell surface receptor can be used in imaging methods for the detection and characterization of tumors. For example, overexpression of any of a number of tyrosine kinase cell surface receptors is known to be associated with cancer. Receptor binding analogs and inhibitors are known for a number of these cell surface receptors. Although immunohistochemical analysis of tumor biopsies is common, tumors by their nature are heterogeneous. Therefore, analysis of a small tumor sample may not provide a clear understanding of the characteristics of the tumor. Using the reagents and methods of the invention, a tumor can be imaged as a whole, detecting both the presence or absence of a receptor, but also the heterogeneity of the tumor. FIG. 2 schematically shows an in vivo delivery method to a receptor of interest in the body, e.g. to a specific cell including a specific cell surface receptor. A bispecific ligand including a binding analog or inhibitor of a cell surface receptor and a ligand for binding a payload attached to a polymer including an agent detectable by imaging e.g., a chelator (see. e.g., S. Lui et al, 1997, App. Rad. Isotopes, 48:1103-1111), is administered by infusion. Based on the particular pharmacokinetic and pharmacodynamic properties of the bispecific ligand, the detectable payload (i.e., ⁹⁹Tc) linked to a tether is administered to promote binding of the tether to the bispecific ligand bound at the tumor rather than in circulation. MRI is used to detect the payload bound to the bispecific lesion.

It is understood that a similar method can be used to deliver and agent to a cell in culture expressing a cell surface receptor that binds the ligand on the bispecific ligand.

### Nucleic Acid Detection

Methods for detection of small amounts specific nucleic acid sequences in samples is a time consuming process with most detection methods requiring amplification of the target sequence by the polymerase chain reaction (PCR). The use of PCR methods requires knowledge of two sequences typically at least about 16 nucleotides in length from portions of the target nucleic acid molecule that are sufficiently far apart to form an amplification product. This limits the ability to detect short nucleic acids, e.g., siRNAs, microRNAs, nucleic acid therapeutics, using PCR. Other methods such as northern blotting are time consuming and require large amounts of material, and detection using HPLC is time consuming by requiring isolation of the nucleic acid and a high level of expertise.

FIG. 3 is a schematic of the use of the compositions and methods provided herein for the detection of a nucleic acid in a sample. In the schematic, the nucleic acid is attached to a solid support and denatured. Depending on the sample, the methods for denaturation of the nucleic acid will vary. Such methods of denaturation are well known to those of skill in the art. The sample is contacted with a bispecific ligand including a nucleic acid sequence for hybridization to the target sequence linked to an antibody that binds a payload on a polymer with an active payload for detection, e.g., HRP. The sample is washed to remove unbound bispecific ligand. The sample is then contacted with a mixed payload polymer containing HRP. The sample is then washed to remove unbound mixed payload polymer. An appropriate substrate is added to the sample for detection of the bound HRP.

The method can be performed similarly wherein the nucleic acid molecule is attached to an avidin moiety and the mixed payload polymer includes biotin as the payload.

Therefore the reagents and methods provided herein allow for the detection of small amounts e.g., 10², 10³, 10⁴, 10⁵ molecules of nucleic acid sequences, or nucleic acid sequences present in 1 nm, 100 pM, 10 pM, 1 pM, 100 fM, 10 fM, 1 fM, 100 aM, 10 aM, 1 aM, or less quantities (or any combinations or ranges bracketed by those values), without amplification of the nucleic acid sequence, and without separating the nucleic acid by size from other nucleic acids in the sample by size, or an amplification product from the nucleic acid, based on size.

Mixed payload polymer can include a relatively small active payload, e.g., a fluorophore, radioisotope, in vivo imaging agent, with a larger payload, including an inactive hapten payload, can be used in any of the methods shown schematically in FIGS. 1-3. By capturing the larger payload, the delivery of the smaller active payload to the site of interest is increased, thereby increasing the sensitivity of the detection or diagnostic method.

It is understood that each target binding site is selected independently and can have essentially any specificity as long as one binding site specifically binds a molecule in the sample, and the second binding site does not bind a molecule in the sample. Selection of each binding site will depend, for example, on the sample and the payload. Examples are provided below, but should not be understood to be limiting.

### Target Binding Site Containing Antibodies

In certain embodiments, the compositions and methods of the invention are useful for the diagnosis and treatment of cancer. In such embodiment, the binding site in the bispecific ligand can include an antibody specifically binds to an antigen on a target cell, e.g., a tumor antigen on a tumor cell described herein. Non-limiting examples of tumor antigens include bombesin receptor, HER-2 receptor, EGF receptor, VEGF receptor, gastrin releasing peptide receptor, CEA, AFP, tyrosinase, CA-125, Melan-A/MART-1, NY-CO-38, and NY-ESO-1. Other tumor associated antigens are described in, e.g., Stuass et al., Tumor Antigens Recognized by T Cells and Antibodies, Taylor & Francis (London, 2003); Srinivasan et al., Rev. Recent Clin. Trials 1:283-292 (2006); Simpson et al., Nat. Rev. Cancer 5:615-625 (2005); and U.S. Publ. No. 20060194730.

A number of human monoclonal antibodies against tumor associated antigens, including cell surface, cytoplasmic, and nuclear antigens, have been produced and characterized, and any of these can be used in the compositions and methods described herein (see, e.g., Yoshikawa et al. (1989) Jpn. J. Cancer Res. (Gann) 80:546-553; Yamaguchi et al. (1987) Proc. Natl. Acad. Sci. USA 84:2416-2420; Haspel et al. (1985) Cancer Res. 45:3951-3961; Cote et al. (1986)Proc. Natl. Acad. Sci. USA 83:2959-2963; Glassy (1987) Cancer Res. 47:5181-5188; Borup-Christensen et al. (1987) Cancer Detect. Prevent. Suppl. 1:207-215; Haspel et al. (1985) Cancer Res. 45:3951-3961; Kan-Mitchell et al. (1989) Cancer Res. 49:4536-4541; Yoshikawa et al. (1986) Jpn. J. Cancer Res. 77:1122-1133; and McKnight et al. (1990) Human Antibod. Hybridomas 1:125-129). Other human monoclonal antibodies are described in Olsson (1985) J. Nat. Cancer Inst. 75:397-404; Larrick and Bourla (1986) J. Biol. Resp. Mod. 5:379-393; McCabe et al. (1988) Cancer Res. 48:4348-4353; Research News (1993) Science 262:841; Ditzel et al. (1994) Cancer 73:858-863; Alonso (1991) Am. J. Clin. Oncol. 4:463-471; and Mack et al. (1995) Proc. Natl. Acad. Sci. USA 92:7021-7025. One exemplary antibody useful in the methods described herein is the pan cancer antibody 2C5 (see, e.g., Iakoubov et al., Oncol. Res. 9:439-446 (1997)). It is understood that the specific format of the monoclonal antibodies can be modified to provide alternate antibody formats while retaining binding specificity. Methods to prepare such antibodies are well known to those of skill in the art.

Antibodies to detectable labels for use as payloads, e.g., peroxidases, phosphatases, luciferase, avidin moieties, and biotin are well known and commercially available.

### Target Binding Site Containing Non-Antibody Peptides

In certain embodiments, the bispecific ligands provided herein include a non-antibody, peptide based binding sites to bind the target ligand in the sample to deliver the payload of interest. The peptide can specifically bind a cognate binding partner on the target cell, which together form a binding pair. Non-limiting examples of peptide ligands include a hormone, a cytokine, a polypeptide, e.g. bombesin, peptide based-drug and antigen for binding to a cognate receptor or T- or B-cell. In certain embodiments, the non-antibody peptide binding site can include biotin for binding to a payload avidin moiety on the tether, or an avidin moiety for binding a payload biotin moiety on the tether.

### Target Binding Site Containing Small Molecules

In certain embodiments, the bispecific ligands include small molecules for binding to receptors present in the sample, for example, cocaine for binding to the dopamine receptor, bombesin for binding to the bombesin receptor, acetylcholine for binding to the muscarinic receptor, or dopamine for binding to the dopamine receptor. In certain embodiments, the target binding site can include a chelator for binding a metal or radioactive isotope on a mixed payload polymer.

### Target Binding Site Containing Nucleic Acid

Nucleic acids can hybridize to essentially any target sequence. Methods for designing sequence specific probes and hybridization conditions are well known in the art and depend, for example, on the specific type of nucleic acid used. Typically, “Stringent hybridisation conditions” refers to an overnight incubation at 42° C. in a solution comprising 50% formamide, 5×SSC (750 mM NaCl, 75 mM trisodium citrate), 50 mM sodium phosphate (pH 7.6), 5×Denhardt's solution, 10% dextran sulphate, and 20 pg/ml denatured, sheared salmon sperm DNA, followed by washing the solid supports, typically filters, in 0.1×SSC at about 65° C. It is understood that specific binding conditions can be modified by those of skill in the art based on, for example, the specific composition and length of the probe. In certain embodiments, the tether can include nucleic acid sequences for binding to nucleic acids on the dual specific ligand.

### Methods of Making Bispecific Ligands

A bispecific ligand can be generated by coupling a ligand containing a first target binding site to a second ligand containing a second target binding. For example, an antibody or antibody portion can be functionally linked (e.g., by chemical coupling, genetic fusion, non-covalent association or otherwise) to one or more other molecular entities, such as another antibody or a ligand as described herein. Non-limiting examples of crosslinkers that can be used for chemical coupling include those that are heterobifunctional, having two distinctly reactive groups separated by an appropriate spacer (e.g., m-maleimidobenzoyl-N-hydroxysuccinimide ester) or homobifunctional (e.g., disuccinimidyl suberate). Such linkers are available from, e.g., Pierce Chemical Company, Rockford, Ill. Linkers of different lengths can be selected based on the specific ligands to be joined.

In general, equimolar concentrations of one binding partner will be linked to the other binding partner via covalent bonds as described herein. However, multimeric bispecific complexes may also be generated to improve the avidity of the bispecific complexes, which will provide better targeting molecules.

Methods of synthesis and purification, such as size exclusion chromatograph, can be used to select various bispecific ligands with differ numbers of binding sites for the target and binding sites for the payload. The specific methods to be used will depend on the sizes of the components of the bispecific ligand and the numbers of each of the components desired in the final molecule. Such methods are known and can be selected by those of skill in the art.

In certain embodiments, bispecific ligands can include binding sites for two different targets present on a single cell type, in addition to the binding site to bind the payload-containing tether. The use of binding sites to two different targets on a single cell could increase targeting to cells expressing both targets.

Methods of coupling are known in the art and can result in, e.g., disulfide bonds, thioether bonds, peptide bonds, or ester bonds between the two antibodies or between the antibody and the ligand. Specific methods are described in, e.g., U.S. Pat. No. 6,451,980; Segal et al., Unit 2.13 in Current Protocols in Immunology, John Wiley & Sons, Inc (2003); Sen et al., J. Hum. Stem Cell Res. 10:247-260 (2001); and Bernatowicz et al., Anal. Biochem. 155:95-102 (1986)).

Methods of mixing peptides with corresponding “knobs” and “holes” are known in the art (see, e.g., U.S. Pat. No. 5,582,996).

Expression vectors can be generated for the tandem expression of one or more CDR and/or heavy chain and light chain variable domains for expression of variable domains and/or scFvs with appropriate linkers to permit pairing between complementary CDRs and/or heavy variable chains with light variable chains while preventing the pairing of non-complementary antigen binding domain sequences.

### Tethers in Mixed Payload Polymers

A number of types of polymers can be used as tethers for payload, and optionally other molecules, which bind to a bispecific ligands provided herein. In some instances, polycation payload containing tethers are used in the methods described herein. Such polycation polymers include, without limitation, poly(allylamine), poly(dimethyldiallyammonim chloride) polylysine, poly(ethylenimine), poly(allylamine), and natural polycations such as dextran amine, polyarginine, chitosan, gelatine A, and/or protamine sulfate. In other instances, polyanion polymers are used, including, without limitation, poly(styrenesulfonate), polyglutamic or alginic acids, poly(acrylic acid), poly(aspartic acid), poly(glutaric acid), and natural polyelectrolytes with similar ionized groups such as dextran sulfate, carboxymethyl cellulose, hyaluronic acid, sodium alginate, gelatine B, chondroitin sulfate, and/or heparin. These polymers can be synthesized using known methods, isolated from natural sources, or, in some cases, commercially obtained.

In certain instances, biodegradable and/or biocompatible polymers are used. These include, without limitation, substantially pure carbon lattices (e.g., graphite), dextran, polysaccharides, polypeptides, polynucleotides, acrylate gels, polyanhydride, poly(lactide-co-glycolide), polytetrafluoroethylene, polyhydroxyalkonates, cross-linked alginates, gelatin, collagen, cross-linked collagen, collagen derivatives (such as succinylated collagen or methylated collagen), cross-linked hyaluronic acid, chitosan, chitosan derivatives (such as methylpyrrolidone-chitosan), cellulose and cellulose derivatives (such as cellulose acetate or carboxymethyl cellulose), dextran derivatives (such carboxymethyl dextran), starch and derivatives of starch (such as hydroxyethyl starch), other glycosaminoglycans and their derivatives, other polyanionic polysaccharides or their derivatives, polylactic acid (PLA), polyglycolic acid (PGA), a copolymer of a polylactic acid and a polyglycolic acid (PLGA), lactides, glycolides, and other polyesters, polyglycolide homopolymers, polyoxanones and polyoxalates, copolymer of poly(bis(p-carboxyphenoxy)propane)anhydride (PCPP) and sebacic acid, poly(l-glutamic acid), poly(d-glutamic acid), polyacrylic acid, poly(dl-glutamic acid), poly(l-aspartic acid), poly(d-aspartic acid), poly(dl-aspartic acid), polyethylene glycol, copolymers of the above listed polyamino acids with polyethylene glycol, polypeptides, such as, collagen-like, silk-like, and silk-elastin-like proteins, polycaprolactone, poly(alkylene succinates), poly(hydroxy butyrate) (PHB), polybutylene diglycolate), nylon-2/nylon-6-copolyamides, polydihydropyrans, polyphosphazenes, poly(ortho ester), poly(cyano acrylates), polyvinylpyrrolidone, polyvinylalcohol, poly casein, keratin, myosin, and fibrin, silicone rubbers, or polyurethanes, and the like. Other biodegradable materials that can be used include naturally derived polymers, such as acacia, gelatin, dextrans, albumins, alginates/starch, and the like; or synthetic polymers, whether hydrophilic or hydrophobic.

Other payload containing tethers include dendrimers, liposomes, long circulating liposomes, micelles, nano-molecules, nano-particles, macromolecules, vesicles, and any molecule that can be modified with drugs for diagnosis or therapy, and others. These vesicles and particles can be synthesized using known methods, isolated from natural sources, or, in some cases, are commercially available.

Provided herein are mixed payload polymers that include at least one of each of at least two distinct payload molecules. In certain embodiments, the mixed payload polymers consist essentially of the tether and the payload molecules. That is, the mixed payload polymers do not include other components, particularly other small components, for binding to a specific binding site, e.g., do not include DTPA (or other molecules that are not detectable labels or therapeutic agents) for binding to an anti-DTPA antibody.

In certain embodiments, the mixed payload polymers consist essentially of the tether, the payload molecules, and one or more biotin moieties that are not payload molecules, i.e., are not used for detection of the molecule in the sample. The biotin moiety allows for the multimerization of the mixed payload polymers.

In certain embodiments, the tether is not succinylated or otherwise modified to provide a tether with a negative charge. In certain embodiments, the tether is succinylated or otherwise modified to increase the negative charge. Molecules used for homogeneous modification of the tether to alter charge, solubility, etc., in preferred embodiments are not payload molecules.

In certain embodiments, a binding site of the bispecific ligand binds directly to the payload in the payload-containing tether. In certain embodiments, the bispecific ligand binds to an active payload. In certain embodiments, the bispecific ligand binds to an inactive, hapten payload, e.g., PEG. In certain embodiments, the hapten is a larger payload on the tether. In certain embodiments, the hapten is the largest payload on the tether.

In a preferred embodiment, binding of the mixed payload polymers does not inhibit the activity of the payload molecules on the tether not directly bound to the bispecific ligand. That is, the activity of the payload molecules is inhibited 30% or less, 25% or less, 20% or less, 15% or less, 10% or less, or 5% or less by the binding of the payload containing tether to the bispecific ligand. It is understood that the activity of the payload in the binding site of the bispecific ligand may be completely or nearly completely inhibited (e.g., at least 80%, at least 85%, at least 90%, at least 95%, or more).

The methods described herein are not limited by the particular antigen coupled to the payload-containing tether, provided that the antibody can specifically bind to such antigen. Non-limiting examples of antigens include diethylene triaminepentaacetic acid (DTPA), ethylene diamine tetraacetic acid (EDTA), dinitrophenol, and 1,4,7,10-tetraazacyclododecane-N,N′,N″,N′″-tetraacetic acid (DOTA). However, in certain embodiments, the tethers do not include one or more of, or any of, diethylene triaminepentaacetic acid (DTPA), ethylene diamine tetraacetic acid (EDTA), dinitrophenol, and 1,4,7,10-tetraazacyclododecane-N,N′,N″,N′″-tetraacetic acid (DOTA). Other examples of antigens can be small drug molecules, such as doxorubicin, aspirin, tamoxifen, paclitaxel, which can function as haptens on carriers to generate specific anti-hapten antibodies. The antigen can be coupled to the mixed payload polymers using methods described herein, e.g., by chemical coupling. It is understood that such drugs can act as “payload” molecules when the drug is the material to be delivered to the site of interest.

The mixed payload polymers can be radiolabeled using techniques known in the art. In some situations, a payload containing tether described herein is contacted with a chelating agent, e.g., 1,4,7,10-tetraazacyclododecane-N,N′,N″,N′″-tetraacetic acid (DOTA), to thereby produce a conjugated payload containing tether. The conjugated payload containing tether is then radiolabeled with a radioisotope, e.g., ¹¹¹In, ⁹⁰Y, ¹⁷⁷Lu, ¹⁸⁶Re, ¹⁸⁷Re, or ⁹⁹mTc, to thereby produce a labeled payload containing tether. In other methods, the payload containing tethers can be labeled with ¹¹¹In and ⁹⁰Y using weak transchelators such as citrate (see, e.g., Khaw et al., Science 209:295-297 (1980)) or ⁹⁹mTc after reduction in reducing agents such as Na Dithionite (see, e.g., Khaw et al., J. Nucl. Med. 23:1011-1019 (1982)) or by SnCl₂ reduction (see, e.g., Khaw et al., J. Nucl. Med. 47:868-876 (2006)). Other methods are described in, e.g., Lindegren et al., Bioconjug. Chem. 13:502-509 (2002); Boyd et al., Mol. Pharm. 3:614-627 (2006); and del Rosario et al., J. Nucl. Med. 34:1147-1151 (1993).

### Therapeutic Agents

In some methods described herein, the mixed payload polymer used is conjugated to a therapeutic agent. For example, the therapeutic agent can be a therapeutically active radioisotope described above. Non-limiting examples of other therapeutic agents include antimetabolites (e.g., methotrexate, 6-mercaptopurine, 6-thioguanine, cytarabine, 5-fluorouracil decarbazine), alkylating agents (e.g., mechlorethamine, thioepa chlorambucil, CC-1065, melphalan, carmustine (BSNU), lomustine (CCNU), cyclothosphamide, busulfan, dibromomannitol, streptozotocin, mitomycin, and cis-dichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine, vinblastine, taxol, paclitaxel, and maytansinoids). Other therapeutic agents include, e.g., cytochalasin B, gramicidin D, ethidium bromide, emetine, etoposide, tenoposide, colchicin, dihydroxy anthracin dione, mitoxantrone, mithramycin, 1-dehydrotestosterone, glucocorticoids, procaine, tetracaine, lidocaine, propranolol, puromycin, calicheamicin, tamoxifin, paclitexal, and analogs or homologs thereof.

In particular instances, the therapeutic agent is non-toxic, or exhibits reduced toxicity at the effective dose, when conjugated to the tether. Without being bound by theory, it is believed that upon binding of a therapeutic agent-conjugated tether to a bispecific binding complex (which is itself specifically bound to a target cell), the therapeutic agent-conjugated tether is internalized by the cell. Upon entry to the cell, the therapeutic agent is released from the tether, typically as a result of intracellular enzymes, and regains its toxicity. Thus, using the methods described herein, cells can be targeted with increased safety.

### Mixed Payload Polymers

The compositions and methods provided herein can include the use of mixed payload polymers. For example, a number of therapeutic regimens for the treatment of cancer include administration of a combination of therapeutic agents. The polymer containing the mixture of therapeutic agents can be delivered to the site of interest, e.g., a tumor, using a bispecific ligand that binds the tumor in one binding site and binds a therapeutic agent at the other binding site. It is understood that not all drugs for use in the regimen need to be delivered on a single tether.

The compositions and methods provided herein can include the use of mixed payload tethers including both therapeutic agents and in vivo diagnostic agents. A tether including one or more therapeutic agents with one or more in vivo diagnostic agents, particularly agents of about the same molecular weight. The polymer containing the mixture of agents can be delivered to the site of interest, e.g., a tumor, using a bispecific ligand that binds the tumor in one binding site and binds the therapeutic agent at the other binding site. Such agents could be particularly advantageous for the treatment, monitoring, and detection of metastatic disease with a single agent.

In certain embodiments, the compositions and methods can include the mixtures of mixed payload polymers that all contain the same inactive hapten for capture by the bispecific ligand, e.g., PEG, and include various active payloads or mixtures of active payloads on each tether.

The compositions and methods provided herein can include the use of mixed payload polymers including a plurality of payloads for in vitro detection methods. A tether including one or more payloads for in vitro detection methods, particularly payloads wherein at least one of the distinct payload molecules, or all of the active payload molecules are smaller than the hapten for capture by the bispecific ligand. The polymer containing the mixture of payloads can be delivered to the bispecific ligand bound to the target attached to a solid support. The polymer with mixed payload tethers may include, for example, a fluorophore and biotin or PEG to permit the use of a single reagent across in vitro detection methods, e.g., immunofluorescence and ELISA, or to permit the detection or quantitation of the payload using different detection methods, fluorescence and enzymatic.

### Diseases/Disorders

The compositions and methods provided herein can be used to inhibit the growth, progression, and/or metastasis of hyperproliferative, hyperplastic, metaplastic, dysplastic, and pre-neoplastic diseases or disorders; or any other disease or disorder that would benefit from the targeted delivery of a therapeutic. Similarly, the compositions and methods provided herein can be used for the diagnosis and monitoring of hyperproliferative, hyperplastic, metaplastic, dysplastic, and pre-neoplastic diseases or disorders; or any other disease or disorder that would benefit from the sensitive imaging and diagnostic methods provided herein.

“Hyperproliferative disease or disorder” is understood as a neoplastic cell growth or proliferation, whether malignant or benign, including all transformed cells and tissues and all cancerous cells and tissues. Hyperproliferative diseases or disorders include, but are not limited to, precancerous lesions, abnormal cell growths, benign tumors, malignant tumors, and cancer. Additional non-limiting examples of hyperproliferative diseases, disorders, and/or conditions include neoplasms, whether benign or malignant, located in the prostate, colon, abdomen, bone, breast, digestive system, liver, pancreas, peritoneum, endocrine glands (adrenal, parathyroid, pituitary, testicles, ovary, thymus, thyroid), eye, head and neck, nervous (central and peripheral), lymphatic system, pelvic, skin, soft tissue, spleen, thoracic, or urogenital tract.

As used herein, the term “tumor” or “tumor tissue” refers to an abnormal mass of tissue that results from excessive cell division. A tumor or tumor tissue comprises “tumor cells”, which are neoplastic cells with abnormal growth properties and no useful bodily function. Tumors, tumor tissue, and tumor cells may be benign or malignant. A tumor or tumor tissue can also comprise “tumor-associated non-tumor cells”, such as vascular cells that form blood vessels to supply the tumor or tumor tissue. Non-tumor cells can be induced to replicate and develop by tumor cells, for example, induced to undergo angiogenesis within or surrounding a tumor or tumor tissue.

As used herein, “malignancy” is understood as a non-benign tumor or a cancer. As used herein, the term “cancer” means a type of hyperproliferative disease that includes a malignancy characterized by deregulated or uncontrolled cell growth. Examples of cancer include, but are not limited to, carcinoma, lymphoma, blastoma, sarcoma, and leukemia or lymphoid malignancies. More particular examples of such cancers are noted below and include squamous cell cancer (e.g., epithelial squamous cell cancer), lung cancer (including small-cell lung cancer, non-small cell lung cancer, adenocarcinoma of the lung and squamous carcinoma of the lung), cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial cancer, uterine carcinoma, salivary gland carcinoma, kidney or renal cancer, prostate cancer, vulval cancer, thyroid cancer, hepatic carcinoma, anal carcinoma, penile carcinoma, as well as head and neck cancer. The term “cancer” includes primary malignant cells or tumors (e.g., those whose cells have not migrated to sites in the subject's body other than the site of the original malignancy or tumor) and secondary malignant cells or tumors (e.g., those arising from metastasis, the migration of malignant cells or tumor cells to secondary sites that are different from the site of the original tumor).

Other examples of cancers or malignancies include, but are not limited to, Acute Childhood Lymphoblastic Leukemia, Acute Lymphoblastic Leukemia, Acute Lymphocytic Leukemia, Acute Myeloid Leukemia, Adrenocortical Carcinoma, Adult (Primary) Hepatocellular Cancer, Adult (Primary) Liver Cancer, Adult Acute Lymphocytic Leukemia, Adult Acute Myeloid Leukemia, Adult Hodgkin's Disease, Adult Hodgkin's Lymphoma, Adult Lymphocytic Leukemia, Adult Non-Hodgkin's Lymphoma, Adult Primary Liver Cancer, Adult Soft Tissue Sarcoma, AIDS-Related Lymphoma, AIDS-Related Malignancies, Anal Cancer, Astrocytoma, Bile Duct Cancer, Bladder Cancer, Bone Cancer, Brain Stem Glioma, Brain Tumors, Breast Cancer, Cancer of the Renal Pelvis and Ureter, Central Nervous System (Primary) Lymphoma, Central Nervous System Lymphoma, Cerebellar Astrocytoma, Cerebral Astrocytoma, Cervical Cancer, Childhood (Primary) Hepatocellular Cancer, Childhood (Primary) Liver Cancer, Childhood Acute Lymphoblastic Leukemia, Childhood Acute Myeloid Leukemia, Childhood Brain Stem Glioma, Childhood Cerebellar Astrocytoma, Childhood Cerebral Astrocytoma, Childhood Extracranial Germ Cell Tumors, Childhood Hodgkin's Disease, Childhood Hodgkin's Lymphoma, Childhood Hypothalamic and Visual Pathway Glioma, Childhood Lymphoblastic Leukemia, Childhood Medulloblastoma, Childhood Non-Hodgkin's Lymphoma, Childhood Pineal and Supratentorial Primitive Neuroectodermal Tumors, Childhood Primary Liver Cancer, Childhood Rhabdomyosarcoma, Childhood Soft Tissue Sarcoma, Childhood Visual Pathway and Hypothalamic Glioma, Chronic Lymphocytic Leukemia, Chronic Myelogenous Leukemia, Colon Cancer, Cutaneous T-Cell Lymphoma, Endocrine Pancreas Islet Cell Carcinoma, Endometrial Cancer, Ependymoma, Epithelial Cancer, Esophageal Cancer, Ewing's Sarcoma and Related Tumors, Exocrine Pancreatic Cancer, Extracranial Germ Cell Tumor, Extragonadal Germ Cell Tumor, Extrahepatic Bile Duct Cancer, Eye Cancer, Female Breast Cancer, Fibrosarcoma, Gaucher's Disease, Gallbladder Cancer, Gastric Cancer, Gastrointestinal Carcinoid Tumor, Gastrointestinal Tumors, Germ Cell Tumors, Gestational Trophoblastic Tumor, Hairy Cell Leukemia, Head and Neck Cancer, Hepatocellular Cancer, Hodgkin's Disease, Hodgkin's Lymphoma, Hypergammaglobulinemia, Hypopharyngeal Cancer, Intestinal Cancers, Intraocular Melanoma, Islet Cell Carcinoma, Islet Cell Pancreatic Cancer, Kaposi's Sarcoma, Kidney Cancer, Laryngeal Cancer, Lip and Oral Cavity Cancer, Liver Cancer, Lung Cancer, Lymphoproliferative Disorders, Macroglobulinemia, Male Breast Cancer, Malignant Mesothelioma, Malignant Thymoma, Medulloblastoma, Melanoma, Mesothelioma, Metastatic Occult Primary Squamous Neck Cancer, Metastatic Primary Squamous Neck Cancer, Metastatic Squamous Neck Cancer, Multiple Myeloma, Multiple Myeloma/Plasma Cell Neoplasm, Myelodysplastic Syndrome, Myelogenous Leukemia, Myeloid Leukemia, Myeloproliferative Disorders, Nasal Cavity and Paranasal Sinus Cancer, Nasopharyngeal Cancer, Neuroblastoma, Non-Hodgkin's Lymphoma During Pregnancy, Nonmelanoma Skin Cancer, Non-Small Cell Lung Cancer, Occult Primary Metastatic Squamous Neck Cancer, Oropharyngeal Cancer, Osteo-/Malignant Fibrous Sarcoma, Osteosarcoma/Malignant Fibrous Histiocytoma, Osteosarcoma/Malignant Fibrous Histiocytoma of Bone, Ovarian Epithelial Cancer, Ovarian Germ Cell Tumor, Ovarian Low Malignant Potential Tumor, Pancreatic Cancer, Paraproteinemias, Purpura, Parathyroid Cancer, Penile Cancer, Pheochromocytoma, Pituitary Tumor, Plasma Cell Neoplasm/Multiple Myeloma, Primary Central Nervous System Lymphoma, Primary Liver Cancer, Prostate Cancer, Rectal Cancer, Renal Cell Cancer, Renal Pelvis and Ureter Cancer, Retinoblastoma, Rhabdomyosarcoma, Salivary Gland Cancer, Sarcoidosis Sarcomas, Sezary Syndrome, Skin Cancer, Small Cell Lung Cancer, Small Intestine Cancer, Soft Tissue Sarcoma, Squamous Neck Cancer, Stomach Cancer, Supratentorial Primitive Neuroectodermal and Pineal Tumors, T-Cell Lymphoma, Testicular Cancer, Thymoma, Thyroid Cancer, Transitional Cell Cancer of the Renal Pelvis and Ureter, Transitional Renal Pelvis and Ureter Cancer, Trophoblastic Tumors, Ureter and Renal Pelvis Cell Cancer, Urethral Cancer, Uterine Cancer, Uterine Sarcoma, Vaginal Cancer, Visual Pathway and Hypothalamic Glioma, Vulvar Cancer, Waldenstrom's Macroglobulinemia, and Wilm's Tumor.

The compositions and methods provided herein can also be used to treat premalignant conditions and to prevent progression to a neoplastic or malignant state including, but not limited to, those disorders described above. Such uses are indicated in conditions known or suspected of preceding progression to neoplasia or cancer, in particular where non-neoplastic cell growth consisting of hyperplasia, metaplasia, or dysplasia has occurred (see, e.g., Robbins and Angell, Basic Pathology, 2d Ed., W.B. Saunders Co., Philadelphia, pp. 68-79 (1976)).

The compositions and methods provided herein can further be used to treat hyperplastic disorders. Hyperplasia is a form of controlled cell proliferation, involving an increase in cell number in a tissue or organ, without significant alteration in structure or function. Hyperplastic disorders include, but are not limited to, angiofollicular mediastinal lymph node hyperplasia, angiolymphoid hyperplasia with eosinophilia, atypical melanocytic hyperplasia, basal cell hyperplasia, benign giant lymph node hyperplasia, cementum hyperplasia, congenital adrenal hyperplasia, congenital sebaceous hyperplasia, cystic hyperplasia, cystic hyperplasia of the breast, denture hyperplasia, ductal hyperplasia, endometrial hyperplasia, fibromuscular hyperplasia, focal epithelial hyperplasia, gingival hyperplasia, inflammatory fibrous hyperplasia, inflammatory papillary hyperplasia, intravascular papillary endothelial hyperplasia, nodular hyperplasia of prostate, nodular regenerative hyperplasia, pseudoepitheliomatous hyperplasia, senile sebaceous hyperplasia, and verrucous hyperplasia.

The compositions and methods provided herein can also be used to treat metaplastic disorders. Metaplasia is a form of controlled cell growth in which one type of adult or fully differentiated cell substitutes for another type of adult cell. Metaplastic disorders include, but are not limited to, agnogenic myeloid metaplasia, apocrine metaplasia, atypical metaplasia, autoparenchymatous metaplasia, connective tissue metaplasia, epithelial metaplasia, intestinal metaplasia, metaplastic anemia, metaplastic ossification, metaplastic polyps, myeloid metaplasia, primary myeloid metaplasia, secondary myeloid metaplasia, squamous metaplasia, squamous metaplasia of amnion, and symptomatic myeloid metaplasia.

The compositions and methods provided herein can also be used to treat dysplastic disorders. Dysplasia can be a forerunner of cancer and is found mainly in the epithelia. Dysplasia is a disorderly form of non-neoplastic cell growth, involving a loss in individual cell uniformity and in the architectural orientation of cells. Dysplastic cells can have abnormally large, deeply stained nuclei, and exhibit pleomorphism. Dysplasia can occur, e.g., in areas of chronic irritation or inflammation. Dysplastic disorders include, but are not limited to, anhidrotic ectodermal dysplasia, anterofacial dysplasia, asphyxiating thoracic dysplasia, atriodigital dysplasia, bronchopulmonary dysplasia, cerebral dysplasia, cervical dysplasia, chondroectodermal dysplasia, cleidocranial dysplasia, congenital ectodermal dysplasia, craniodiaphysial dysplasia, craniocarpotarsal dysplasia, craniometaphysial dysplasia, dentin dysplasia, diaphysial dysplasia, ectodermal dysplasia, enamel dysplasia, encephalo-ophthalmic dysplasia, dysplasia epiphysialis hemimelia, dysplasia epiphysialis multiplex, dysplasia epiphysialis punctata, epithelial dysplasia, faciodigitogenital dysplasia, familial fibrous dysplasia of the jaws, familial white folded dysplasia, fibromuscular dysplasia, fibrous dysplasia of bone, florid osseous dysplasia, hereditary renal-retinal dysplasia, hidrotic ectodermal dysplasia, hypohidrotic ectodermal dysplasia, lymphopenic thymic dysplasia, mammary dysplasia, mandibulofacial dysplasia, metaphysial dysplasia, Mondini dysplasia, monostotic fibrous dysplasia, mucoepithelial dysplasia, multiple epiphysial dysplasia, oculoauriculovertebral dysplasia, oculodentodigital dysplasia, oculovertebral dysplasia, odontogenic dysplasia, opthalmomandibulomelic dysplasia, periapical cemental dysplasia, polyostotic fibrous dysplasia, pseudoachondroplastic spondyloepiphysial dysplasia, retinal dysplasia, septo-optic dysplasia, spondyloepiphysial dysplasia, and ventriculoradial dysplasia.

Additional pre-neoplastic disorders that can be treated by the compositions and methods described herein include, but are not limited to, benign dysproliferative disorders (e.g., benign tumors, fibrocystic conditions, tissue hypertrophy, intestinal polyps, colon polyps, and esophageal dysplasia), leukoplakia, keratoses, Bowen's disease, Farmer's Skin, solar cheilitis, and solar keratosis.

Conditions other than proliferative disorders that can be treated include diseases and conditions that are confined to a specific tissue in the body to which a bispecific ligand can be targeted, e.g., liver antigens for hepatitis, cartilage for arthritis, heart for cardiac diseases, fibroids in various tissues.

The compositions and methods described herein can also be used as diagnostic agents for conditions other than proliferative disorders, e.g., for plaque formation, atherosclerosis, fibrosis, arthritis, infection.

### Pharmaceutical Compositions and Administration

The bispecific ligands and mixed payload polymers provided herein can be incorporated into pharmaceutical compositions to be used in the methods described herein. Such compositions can include a pharmaceutically acceptable bispecific ligand and/or pharmaceutically acceptable mixed payload polymer; and a pharmaceutically acceptable carrier.

As used herein, a “pharmaceutically acceptable carrier” means a carrier that can be administered to a subject together with a bispecific ligand and mixed payload polymer provided herein, which does not destroy the pharmacological activity thereof. Pharmaceutically acceptable carriers include, e.g., solvents, dispersion media, coatings, antibacterial and antifungal agents, isotonic and absorption delaying agents, and the like, compatible with pharmaceutical administration. Supplementary active compounds can also be incorporated into the compositions.

Non-limiting examples of pharmaceutically acceptable carriers that can be used include poly(ethylene-co-vinyl acetate), PVA, partially hydrolyzed poly(ethylene-co-vinyl acetate), poly(ethylene-co-vinyl acetate-co-vinyl alcohol), a cross-linked poly(ethylene-co-vinyl acetate), a cross-linked partially hydrolyzed poly(ethylene-co-vinyl acetate), a cross-linked poly(ethylene-co-vinyl acetate-co-vinyl alcohol), poly-D, L-lactic acid, poly-L-lactic acid, polyglycolic acid, PGA, copolymers of lactic acid and glycolic acid (PLGA), polycaprolactone, polyvalerolactone, poly (anhydrides), copolymers of polycaprolactone with polyethylene glycol, copolymers of polylactic acid with polyethylene glycol, polyethylene glycol; and combinations and blends thereof.

Other carriers include, e.g., an aqueous gelatin, an aqueous protein, a polymeric carrier, a cross-linking agent, or a combination thereof. In another instances, the carrier is a matrix. In yet another instances, the carrier includes water, a pharmaceutically acceptable buffer salt, a pharmaceutically acceptable buffer solution, a pharmaceutically acceptable antioxidant, ascorbic acid, one or more low molecular weight pharmaceutically acceptable polypeptides, a peptide comprising about 2 to about 10 amino acid residues, one or more pharmaceutically acceptable proteins, one or more pharmaceutically acceptable amino acids, an essential-to-human amino acid, one or more pharmaceutically acceptable carbohydrates, one or more pharmaceutically acceptable carbohydrate-derived materials, a non-reducing sugar, glucose, sucrose, sorbitol, trehalose, mannitol, maltodextrin, dextrins, cyclodextrin, a pharmaceutically acceptable chelating agent, EDTA, DTPA, a chelating agent for a divalent metal ion, a chelating agent for a trivalent metal ion, glutathione, pharmaceutically acceptable nonspecific serum albumin, and/or combinations thereof.

A pharmaceutical composition containing a bispecific ligand or mixed payload polymer can be formulated to be compatible with its intended route of administration as known by those of ordinary skill in the art. Non-limiting examples of routes of administration include parenteral, intravenous, intradermal, subcutaneous, oral (e.g., inhalation), transdermal (topical), transmucosal, vaginal and rectal administration. Solutions or suspensions used for parenteral, intradermal, or subcutaneous application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. It may be desirable to include isotonic agents, for example, sugars, polyalcohols such as mannitol, sorbitol, or sodium chloride in the composition. Prolonged absorption of the injectable compositions can be accomplished by including in the composition an agent that delays absorption, for example, aluminum monostearate and gelatin (see, e.g., Remington: The Science and Practice of Pharmacy, 21st edition, Lippincott Williams & Wilkins, Gennaro, ed. (2006)).

Sterile injectable solutions can be prepared by incorporating a bispecific ligand or mixed payload polymer in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the methods of preparation include, without limitation, vacuum drying and freeze-drying which yields a powder of the active ingredient plus any additional desired ingredient from a previously sterile-filtered solution thereof.

Oral compositions generally include an inert diluent or an edible carrier. For the purpose of oral therapeutic administration, a bispecific binding complex or mixed payload polymer can be incorporated with excipients and used in the form of tablets, pills, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, a bispecific binding complex or mixed payload polymer can be delivered in the form of an aerosol spray from pressured container or dispenser that contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, but are not limited to, for example, for transmucosal administration, detergents, bile salts, and fusidic acid derivatives. Transmucosal administration can be accomplished through the use of nasal sprays or suppositories. For transdermal administration, the active compounds are formulated into, e.g., ointments, salves, gels, or creams as generally known in the art.

The pharmaceutical compositions containing a bispecific binding complex or mixed payload polymer can also be prepared in the form of suppositories (e.g., with conventional suppository bases such as cocoa butter and other glycerides) or retention enemas for rectal delivery.

Some pharmaceutical compositions can be prepared with a carrier that protects the bispecific binding complex or mixed payload polymer against rapid elimination from the body, such as a controlled release formulation, including implants and microencapsulated delivery systems (as described, e.g., in Tan et al., Pharm. Res. 24:2297-2308, 2007). Biodegradable, biocompatible polymers can be used, such as ethylene vinyl acetate, polyanhydrides, polyglycolic acid, collagen, polyorthoesters, and polylactic acid. Methods for preparation of such formulations are apparent to those skilled in the art. The materials can also be obtained commercially (e.g., from Alza Corp., Mountain View, Calif.). Liposomal suspensions (including liposomes with the bispecific binding complex or mixed payload polymer on their surface) can also be used as pharmaceutically acceptable carriers. These can be prepared according to methods known to those skilled in the art, e.g., as described in U.S. Pat. No. 4,522,811.

It may be advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier.

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. While compounds that exhibit toxic side effects can be used, care should be taken to design a delivery system that targets such compounds to the site of affected tissue in order to minimize potential damage to uninfected cells and, thereby, reduce side effects. The invention includes the use of the compositions and methods provided herein to reduce the toxicity of the effective dose of a therapeutic or imaging agent, or increase the therapeutic index of a therapeutic or imaging agent.

The data obtained from the cell culture assays and animal studies can be used in formulating a range of dosage for use in humans. The dosage of such compounds lies generally within a range of circulating concentrations that include the ED₅₀ with little or no toxicity. The dosage can vary within this range depending upon the dosage form employed and the route of administration utilized. For any compound used in the method of the invention, the therapeutically effective dose can be estimated initially from cell culture assays. A dose can be formulated in animal models to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma can be measured, for example, by high performance liquid chromatography. Information for preparing and testing such compositions are known in the art (see, e.g., Remington's The Science and Practice of Pharmacy, 21st edition, Lippincott Williams & Wilkins, Gennaro, ed. (2006)).

In some instances, a therapeutically effective amount or dosage of a bispecific binding complex or mixed payload polymer can range from about 0.001 mg/kg body weight to about 100 mg/kg body weight, e.g., from about 0.01 mg/kg body weight to about 50 mg/kg body weight, from about 0.025 mg/kg body weight to about 25 mg/kg body weight, from about 0.1 mg/kg body weight to about 20 mg/kg body weight, from about 0.25 mg/kg body weight to about 20 mg/kg body weight, from about 0.5 mg/kg body weight to about 20 mg/kg body weight, from about 0.5 mg/kg body weight to about 10 mg/kg body weight, from about 1 mg/kg body weight to about 10 mg/kg body weight, or about 5 mg/kg body weight.

In other instances, a therapeutically effective amount or dosage of a bispecific binding complex or mixed payload polymer can range from about 0.001 mg to about 50 mg total, e.g., from about 0.01 mg to about 40 mg total, from about 0.025 mg to about 30 mg total, from about 0.05 mg to about 20 mg total, from about 0.1 mg to about 10 mg total, or from about 1 mg to about 10 mg total.

A physician will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of a bispecific binding complex and payload containing tether can include a single treatment or a series of treatments. In one example, a subject is treated with a bispecific binding complex and payload containing tether in the range of between about 0.06 mg to 120 mg, one time per week for between about 1 to 10 weeks, alternatively between 2 to 8 weeks, between about 3 to 7 weeks, or for about 4, 5, or 6 weeks. It will also be appreciated that the effective dosage of a bispecific binding complex and mixed payload polymer used for treatment may increase or decrease over the course of a particular treatment.

In particular instances, a bispecific binding complex is administered first, followed by administration of a mixed payload polymer described herein. For example, a bispecific binding complex can be administered first and the mixed payload polymer is subsequently administered 4 hrs later, 8 hrs later, 12 hrs later, 16 hrs later, 20 hrs later, 24 hrs later, 36 hrs later, 48 hrs later, 72 hrs later, or 4 days, 5 days, 6 days, 7 days, or more days, later.

The pharmaceutical compositions can be included in a container, pack, or dispenser together with instructions for administration.

A person of ordinary skill in the art will appreciate that the pharmaceutical compositions described herein can be formulated as single-dose vials. For example, single-dose vials can be produced containing about 25 μg, about 40 μg, about 60 μg, about 100 μg, about 150 μg, about 200 μg, about 300 μg, or about 500 μg of a bispecific binding complex or mixed payload polymer containing pharmaceutical composition described herein. In a further example, single-dose vials can be produced containing a concentration of about 0.5 mM or about 1.0 mM of a pharmaceutical composition described herein.

Treatment of a subject with a therapeutically effective amount of a bispecific binding complex or mixed payload polymer containing pharmaceutical composition described herein can be a single treatment, continuous treatment, or a series of treatments divided into multiple doses. The treatment can include a single administration, continuous administration, or periodic administration over one or more years. Chronic, long-term administration can be indicated in many cases. In some instances, a subject is treated for up to one year. In other instances, a subject is treated for up to 6 months. In yet another situation, a subject is treated for up to 100 days. In one example, a subject is treated with a bispecific binding complex and payload containing tether in a time frame of one time per week for between about 1 week to 10 weeks, alternatively between 2 weeks to 8 weeks, between about 3 weeks to 7 weeks, or for about 4 weeks, 5 weeks, or 6 weeks. In other instances, a subject can be treated substantially continuously. In other situations, a subject can be treated once per day, twice per day, once per week, or once per month.

Generally, each formulation is administered in an amount sufficient to ameliorate at least one sign or a symptom of a disorder or condition described herein.

In addition to treating pre-existing disorders, the methods described herein can prevent or slow the onset of such disorders. For example, the bispecific binding complex and mixed payload polymer described herein can be administered for prophylactic applications, e.g., can be administered to a subject susceptible to or otherwise at risk for a disorder. In some instances, a bispecific binding complex and mixed payload polymer can be administered to a subject who has a pre-existing disorder and is susceptible to or otherwise at risk for a further disorder.

Suppression of a disorder can be evaluated by any known methods of measuring whether the disorder or a symptom of the disorder is slowed or diminished. Such methods include, e.g., direct observation and indirect evaluation, e.g., by evaluating subjective symptoms or objective physiological indicators.

In some instances, a bispecific ligand and mixed payload polymer described herein are administered in combination with one or more additional therapies, e.g., therapeutic agents useful in the treatment of disorders or conditions described herein. For example, the second therapy can include radiation therapy or chemotherapy.

Reference will now be made in detail to preferred embodiments of the invention. While the invention will be described in conjunction with the preferred embodiments, it will be understood that it is not intended to limit the invention to those preferred embodiments. To the contrary, it is intended to cover alternatives, modifications, and equivalents as may be included within the spirit and scope of the invention as defined by the appended claims.

### Example 1

### Making Mixed Payload Polymers

Methods of making the mixed payload polymers of the invention is within the ability of those of skill in the art. For example, poly-lysine is commercially available from a number of sources. Using Edman degradation, a protein is protected at all lysine amino groups while retaining a free amino terminus and such a modified protein is end-labeled by an amino group-specific reagent (radioiodinated Bolton-Hunter reagent). Polyethylene glycol (PEG) can be modified for linkage to a polymer using a number of known methods. Alternatively, PEG can be joined to the peptide resulting in a tether with a 1:1 ratio or tether to analytical tag. Free PEG can be removed by size exclusion chromatography or other methods. The PEG can be added either before or after the payload molecules are attached to the tether. Preferably the poly-lysine molecules are of a known molecular weight, or have a known average molecular weight.

The payload molecules can be attached using any of a number of methods such as those provided in U.S. Pat. No. 6,451,980 or US Patent Publication No. 20090285757, both of which are incorporated herein by reference. Methods for succinylation are also provided therein. Unattached payload molecules are removed from the mixed payload polymers using a method appropriate to the specific payload.

Depending on the particular use of the mixed payload polymers, the mixed payload polymers can be fractionated by size or other methods. The average molecular weight of the mixed payload polymers is determined, or the total amount of material present in the solution containing the mixed payload polymer is determined.

Known synthetic methods can be used to provide populations of tethers with predefined average numbers of payloads. The specific method depends, for example, on the number of payloads, the size of the payloads, the linkage methods for the payloads, and other considerations known in the art. Further, methods of selecting tethers having a predefined average number of payloads are also known in the art.

### Example 2

### Determining the Relative Activity of Tethered Vs Untethered Payloads

For the purpose of simplicity, the payload in this example will be considered to be an enzyme. Similar experiments can be performed in which the payload is a drug using, for example, an apoptosis and/or proliferation assay.

Serial dilutions of equimolar amounts of the payload molecules either free or attached to a tether are aliquoted into wells of a 96-well plate. An appropriate substrate, e.g., a colormetric substrate, is added to each well and the reaction is allowed to proceed for a defined period of time and stopped with the appropriate reagent. The amount of reaction product is determined. The relative activity of the tethered payload molecules is determined on a percent basis by dividing the activity of the tethered payloads by the amount of activity of the non-tethered payloads on an equimolar basis. Such calculations are within the ability of those of ordinary skill in the art.

### Example 3

### Capturing Larger Molecules on a Mixed Payload Polymer Increases Polymer Capture

To demonstrate the improved detection of the target by capturing the payload using a bispecific ligand targeted to the larger payload, rather than smaller payload on the tether, an ELISA style assay was used.

Polylysine tethers joined to both HRP (molecular weight about 44 kDa) and DTPA (molecular weight 393 Da) were synthesized using routine methods and purified using dialysis, HPLC or a spin column.

ELISA plate wells were coated with serial dilutions of one of

1. mouse anti-DTPA antibody (1 μg/ml to 0.001 μ/ml);

2. rabbit anti-HRP antibody (1 μg/ml to 0.001 μ/ml); or

3. donkey serum (negative control).

The wells were washed, blocked, and washed again.

Fifty microliters of the polylysine tethers joined to both HRP and DTPA were added at a 1:100 dilution of the HPLC purified tethers (657 μg/ml) or a 1:10 dilution of the spin column purified (25 μg/ml), and the samples were incubated for 1 hour at 37° C. The wells were washed, and the HRP was detected using K-Blue substrate. The reaction was stopped with 2.5 N HCl and read at 450 nm. The results are shown in the table below:

| | | Antibody Concentration (mg/ml) | | | | |
|---|---|---|---|---|---|---|
| Antibody | Tether Conc. | 1.0 | 0.1 | 0.01 | 0.001 | 0.0001 |
| Mouse anti- | 1:10 (25 μg/ml) | 3.50 | 2.96 | 0.30 | 0.09 | 0.02 |
| DTPA | | | | | | |
| Mouse anti- | 1:100 (657 μg/ml) | 2.38 | 0.39 | 0.02 | 0.00 | 0.00 |
| DTPA | | | | | | |
| Rabbit anti- | 1:10 (25 μg/ml) | 3.49 | 3.29 | 1.76 | 0.40 | 0.04 |
| HRP | | | | | | |
| Rabbit anti- | 1:100 (657 μg/ml) | 3.65 | 2.89 | 0.66 | 0.03 | −0.02 |
| HRP | | | | | | |

A similar experiment was performed in which the HPLC purified tether was diluted 1:50 rather than 1:100. The results were as follows:

| | | Antibody Concentration (mg/ml) | | | | |
|---|---|---|---|---|---|---|
| Antibody | Tether Conc. | 1.0 | 0.1 | 0.01 | 0.001 | 0.0001 |
| Mouse anti- | 1:10 (25 μg/ml) | 3.39 | 2.51 | 0.51 | 0.22 | 0.11 |
| DTPA | | | | | | |
| Mouse anti- | 1:50 (657 μg/ml) | 2.83 | 0.83 | −0.01 | 0.00 | 0.04 |
| DTPA | | | | | | |
| Rabbit anti- | 1:10 (25 μg/ml) | 3.57 | 3.28 | 1.761 | 0.30 | 0.25 |
| HRP | | | | | | |
| Rabbit anti- | 1:50 (657 μg/ml) | 3.55 | 2.72 | 0.82 | 0.55 | 0.20 |
| HRP | | | | | | |

It can be easily observed that when the larger payload (i.e., HRP) is captured, rather than the smaller payload (i.e., DTPA) the amount of signal detected is significantly higher. Without wishing to be bound by theory, it is suggested that the relatively large HRP molecules inhibit binding of the DTPA to the antibody, thereby reducing the signal. By capturing the larger payload, rather than the smaller payload, capture of the payload to the site of interest is increased, and detection levels are decreased.

### Example 4

### Ultrasensitive Detection of Trace Analytes Using Payload Containing Polymers with Varying Numbers of Payload Molecules

Detection of canine cardiac myosin heavy chains was achieved by the methods provided herein using a bispecific ligand that bound the target cardiac myosin heavy chain in serum samples and the payload horseradish peroxidase (HRP). HRP was detected with a chromogen, e.g. orthophenyl-diamine or K-Blue and optical density output signal was read with a standard ELISA reader. Sensitivity was assessed in vitro with a controlled experiment in which the assay was performed with mixed payload polymers loaded with 1.5, 3, 4.5, 6 or 7.5 HRPs, and compared to a conventional competitive inhibition ELISA standard curve.

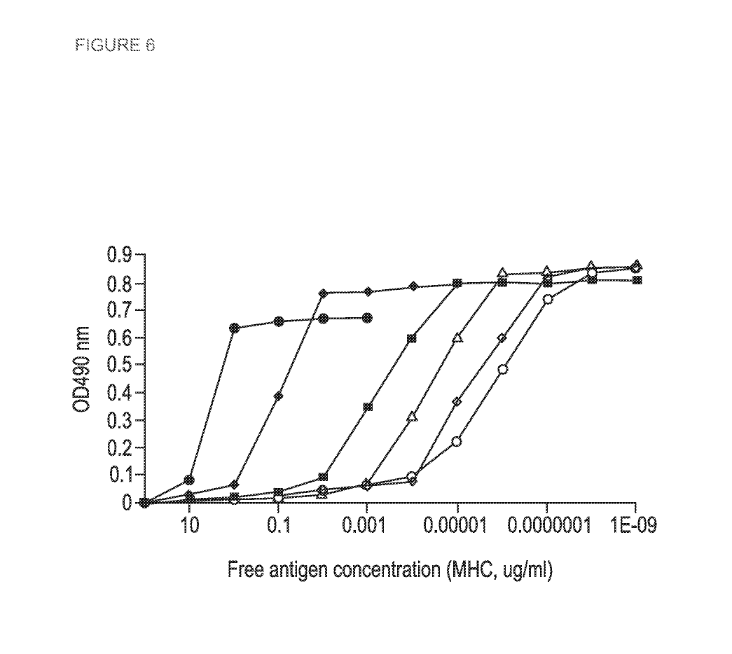

Increasing sensitivity of the standard curves was obtained with increasing HRP loading of payload containing polymers (FIG. 6). The X axis represents the free antigen concentration in the solution for competitive inhibition of the anti-myosin antibody or the anti-myosin bispecific antibody from which 50 μl aliquots were taken for the assays. Therefore 50 μl aliquots of 100 μg/ml canine cardiac myosin contained 1×10⁻¹¹ moles and 50 μl aliquots of 1×10⁻⁸ μg/ml solution contained 1×10⁻²¹ moles. As more HRP molecules were added to the payload containing polymers, the sensitivity steadily increased: starting with 1-HRP ELISA at 1×10⁻¹⁵ moles (FIG. 6, filled circles, far left) and steadily increasing to 7.5 HRPs/payload containing polymer with a sensitivity of 1×10⁻²¹ moles (FIG. 6, open circles, far right). The sensitivity was measured at minus 2 SD of mean maximum binding of antibody or bispecific ligand in the plateau region for each set of assays repeated 3 times. The sensitivity is given at minus 2 SD of mean maximum binding of antibody or bispecific ligand in the plateau region for each set of assays repeated 3 times.

### Example 5

### Detection of Trace Levels of Diagnostic Analytes in Serum Samples

As demonstrated in the prior example, the methods of the invention can be used to detect trace amounts of analytes in samples. The methods provided herein were used for the detection of diagnostic analytes in serum samples.

It is well known that acute myocardial infarction results in the release of cardiac myosin heavy chains into the blood and detection of myosin heavy chains in the blood can assist in the differentiation between acute myocardial infarction and other conditions that may present similar signs and symptoms. The ability to detect low levels of cardiac myosin in the blood would allow for early intervention with appropriate therapies, while avoiding administration of such therapies to subjects not suffering from acute myocardial infarction who may not benefit from, or even be harmed, by such therapies.

Historical serum samples were obtained from subjects who presented in the emergency department of US hospitals with suspected myocardial infarction (MI) who may or may not have finally been diagnosed with a Q-wave or non-Q-wave MI or unstable angina. The samples were analyzed for the presence of cardiac myosin using the methods provided herein and compared to control samples from subjects who did not have MI. Trace amounts of cardiac myosin heavy chain were specifically detected in serum samples from subjects who had been diagnosed with Q-wave and non-Q-wave MI as well as subjects diagnosed with unstable anginal. Cardiac myosin heavy chain was not found in samples from subjects who were not diagnosed with MI. These results demonstrate both the sensitivity of the method and the usefulness of the method to detect analytes in complex samples, e.g., human samples.

Specifically, the bispecific ligand and series of mixed payload polymers described in the prior example used for the detection of cardiac myosin heavy chain fragments in sera from patients with Q-wave MI, non-Q-wave MI, and unstable angina, and compared to control sera. Cardiac myosin heavy chain fragments were detected in the admission blood samples from patients with all forms of MI and unstable angina. However, no detectable levels of cardiac myosin heavy chain fragments were found in control sera. The table below shows the concentration of antigen detected by using the methods herein with payload containing polymers including 1.5, 3, 4.5, and 6 HRP molecules per tether in diluted samples.

| Sera (Dilutions) | 1.5 HRP | 3 HRP | 4.5 HRP | 6 HRP | Mean ± SD/ml |
|---|---|---|---|---|---|
| mg/ml | (non-diluted) | (1/10) | (1/100) | (1/1000) | Serum |
| Q-Wave MI | 2.1 | 1.8E−1 | 1.8E−2 | 2E−3 | 1.93 ± 0.15 |
| Non-Q-Wave MI | 3.5E−3 | 3.2E−4 | 3.8E−5 | 3.0E−6 | 3.4E−3 ± 0.35E−3 |
| Unstable Angina | 2.5E−2 | 1.8E−3 | 2.0E−4 | 2.0E−5 | 2.1E−3 ± 0.30E−2 |

These results demonstrate that, despite the increase in sensitivity from 1.2×10⁻¹⁵ moles to 11.2×10⁻²⁰ moles, the amount of myosin heavy chain fragments detected in each serum sample was essentially the same when corrected for the dilution factor. In conclusion, the compositions and methods provided herein increased the ELISA sensitivity from 10⁻¹³ to 1.5×10⁻²¹ moles of myosin heavy chains. This is equivalent to the detection of less than 1,000 individual molecules per well. The 95% confidence limit, even at this highest sensitivity, was highly significant.

### Example 6

### Comparison of Signal Amplification with Multi-Tagged Reagents

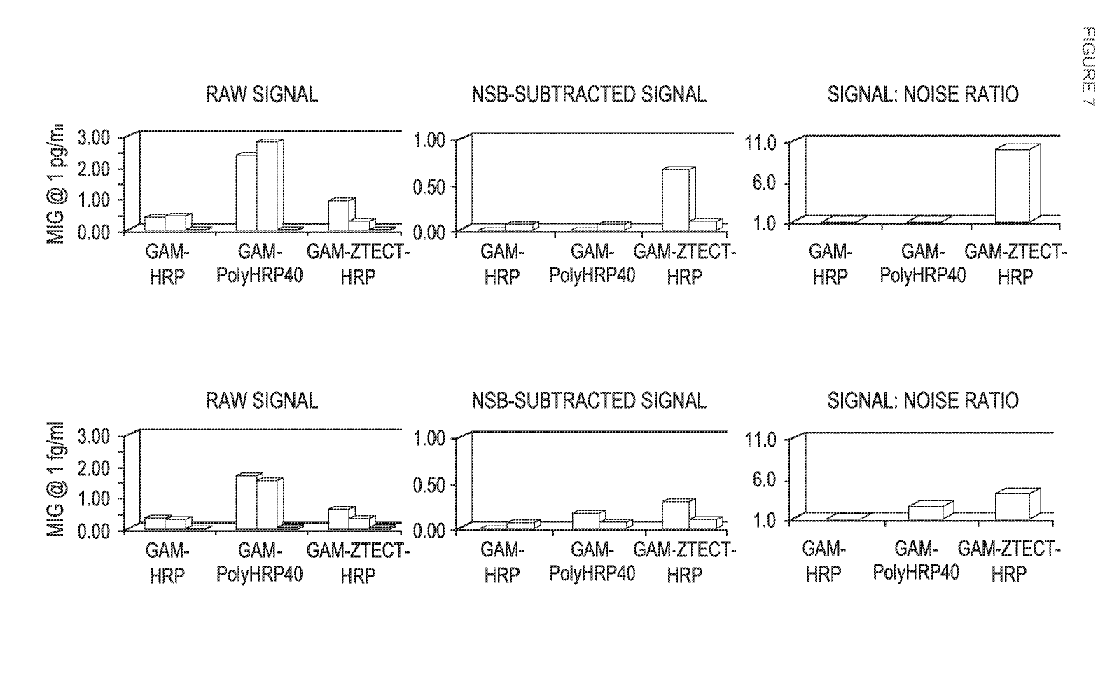

As shown in FIGS. 7A-F to demonstrate the performance of the methods and reagents provided herein, a Goat Anti-Mouse (GAM) antibody from Jackson ImmunoResearch (JIR; West Grove, Pa.) was purchased and used to generate a bispecific GAM-anti-HRP bispecific ligand and for use with an HRP-containing mixed payload polymer. The bispecific ligand and mixed payload polymer detection methods provide herein were tested against the commercial GAM-HRP (also from JIR.) and a commercial GAM-PolyHRP from Thermo Scientific Pierce (Rockford, Ill.) for detecting coated Mouse IgG (MIG) by ELISA. GAM-HRP, GAM-PolyHRP, and GAM-ZTECT-HRP were tested at 1 μg/ml. ELISA plates were coated with 1 pg/ml (top row) and 1 fg/ml (bottom row) of MIG. Data were processed in 2 ways: the Raw Signal data (left column) shows that, although GAM-PolyHRP produces a high OD signal, it also generates very high background (NSB). GAM-HRP underperforms in all cases. The NSB-Subtracted Signal data (middle column) shows that, once background has been subtracted, GAM-PolyHRP does not produce any significant signal. The bispecific ligand and payload containing polymer detection reagents provided herein are the only reagents capable of detecting MIG at 1 pg/ml and 1 fg/ml concentrations. Extrapolating these data confirm that only the bispecific ligand and mixed payload polymer detection reagents provided herein produce a significant signal:noise ratio at 1 pg/ml and 1 fg/ml of MIG. Further, the signal:noise ratio (right column) for the bispecific ligand and mixed payload polymer detection reagents provided herein is 9.6 at 1 pg/ml MIG and still 4.1, even at 1 fg/ml. Since the molecular weight of MIG at 150,000 Daltons (1 Dalton equals 1 g/mol). Since there is 50 μl of MIG in each well, we can calculate that 1 fg/ml MIG corresponds to 50 attograms or 0.33 zeptomoles (10⁻²¹ M) per well. These data show the enhanced detectability of the bispecific ligand and mixed payload polymer detection reagents provided herein achieved with high signal:noise ratios, which are optimal characteristics for all antibody labeling systems.

### Example 7

### Efficiency of Mixed Payload Polymer Detection is Dependent on Captured Payload Size

To confirm that larger payloads resulted in increased capture of polymers in more complex mixed payload polymers, tethers including three distinct payload molecules, HRP (about 44 kDa), PEG (about 20 kDa), and DTPA (393 Da) were generated. Each tether was loaded with about 3-4 HRP molecules, tens of DTPA molecules, and were end labeled with PEG. As in Example 3, ELISA plate wells were coated with one of four antibodies at 10 fold serial dilutions from 10 μg/ml to 0.01 μg/ml.

1. Goat anti-peroxidase (GAP) polyclonal antibody (from US Biological)

2. Goat anti-peroxidase polyclonal antibody (from Jackson Immunoresearch)

3. Mouse anti-DTPA (MAD) monoclonal antibody (from Akrivis)

4. Rabbit anti-PEG monoclonal antibody (from US Biological)

Negative control wells not treated with antibody were used to determine background, and wells untreated with antibody or the mixed payload polymer were used to determine the noise in the assay.

Wells were contacted with equivalent amounts of the mixed payload polymer for a predetermined amount of time and subsequently washed to remove unbound polymers. The wells were reacted with equivalent amounts of peroxidase substrates for a predetermined time period after which the reaction was stopped and the samples were read using a plate reader. All samples were run in triplicate. The results are shown in the table below and in FIG. 9.

| | Average Signal | | | |
|---|---|---|---|---|
| | GAP (USBIO), GAP (JIR), | | | |
| | MAD (AKV), Anti-PEG(USBIO) | | | |
| | 1:100 | 1:1,000 | 1:10,000 | 1:100,000 |
| | 1:100 | 1 ug/ml | 0.1 ug/ml | 0.01 ug/ml |
| GAP (USBIO) | 3.549 | 1.178 | 0.232 | 0.097 |
| St. Dev. | 0.045 | 0.075 | 0.018 | 0.007 |
| GAP (JIR) | 3.535 | 2.949 | 1.456 | 0.195 |
| St. Dev. | 0.060 | 0.031 | 0.068 | 0.008 |
| MAD (AKV) | 0.250 | 0.161 | 0.108 | 0.083 |
| St. Dev. | 0.016 | 0.025 | 0.009 | 0.005 |
| Anti-PEG (USBIO) | 1.299 | 0.327 | 0.117 | 0.104 |
| St. Dev. | 0.019 | 0.012 | 0.010 | 0.009 |
| PEG-H(X)-DPL BKGD | 0.079 | 0.079 | 0.079 | 0.079 |
| NOISE | 0.092 | 0.092 | 0.092 | 0.092 |

As shown, the binding efficiency is highest when the largest payload is captured by the antibody with decreasing efficiency in capturing the polymer with the decreasing size of the payload. This is in spite of the fact that far more of the smallest payload molecules are present on the tether than the larger payload molecules. Therefore, a small number of payload molecules, including inactive payload molecules, can improve the efficiency of delivery of the active payload to the target site.

### Example 8

### Bispecific Ligands with Nucleic Acids for Binding to Targets

Bispecific ligands of the invention include bispecific-molecular probes (BAMPs) which include a nucleic acid for binding to a specific target and a second binding site, typically an antibody, for binding to a payload molecule, e.g., a detectable label. Methods of linking nucleic acids to proteins are well known in the art and can be performed using commercially available reagents and kits. In the methods of the invention, an antibody can be linked to a nucleic acid probe having a specific sequence for binding to a target of interest. Methods to design appropriate control sequences for use are known.

Fluorescence in situ hybridization (FISH) methods are known in the art. Such methods rely on the denaturation of nucleic acids in a tissue sample, typically a tissue section, for detection of nucleic acid sequences in the sample. Such samples can be used in the methods of the invention. Similarly, populations of nucleic acid molecules can be end labeled or modified to allow for the nucleic acids to be linked to a solid support.

The nucleic acids of the sample are contacted with the BAMPs under conditions to allow for hybridization. The specific hybridization conditions will depend on, for example, the length of the nucleic acid and the type and/or modifications in the nucleic acid. The sample is washed to remove unbound BAMPs. Payload containing polymers, including a payload that binds specifically to the non-nucleic acid binding site of the BAMP is added under conditions to allow binding. Unbound payload containing polymers are removed by washing. The sample is reacted with an appropriate substrate to produce a detectable product that is optionally detected quantitatively.

### Example 9

### Bispecific Ligands with Streptavidin for Binding Biotin Containing Tethers

The high affinity binding of biotin to streptavidin is well known and exploited in many biological assays. A tether including multiple biotin moieties is prepared using routine methods such as those provided herein. For example, in certain embodiments, the biotin is synthesized or modified with an N- or C-terminal cross-linking group for attachment to the tether. The cross-linking group is optionally separated from biotin by a peptide linker sequence to that the tether does not interfere with binding of biotin to the streptavidin.

A dual-specific ligand is prepared using any target specific binding agent, for example an antibody, and streptavidin. The sample containing the target molecule is contacted with the dual specific ligand under conditions to permit binding. Unbound dual specific ligand is removed by washing. The sample is subsequently contacted with the biotin-containing tether. The sample is then washed to remove unbound tether. The sample is then contacted with a streptavidin-bound detectable label. The sample is washed to remove unbound streptavidin-bound detectable label.

If the sample is a tissue section or tissue culture slides, or cells for sorting by FACS, the detectable label is a fluorescent label. If the sample is protein bound to a solid support such a nitrocellulose or a well of an ELISA plate, the detectable label is an enzymatic label, for example, horseradish peroxidase.

The method and biotin labeled tether allow for the use of a single dual-specific ligand for detection of the target molecule in multiple types of samples for analysis by multiple methods as the biotin-containing tether that is bound by the streptavidin-containing dual-specific ligand can be bound by any of a number of streptavidin-bound detectable labels.

The method includes binding of the streptavidin-containing dual specific ligand directly to a target molecule endogenous to the sample. The method also includes binding of a streptavidin-containing dual specific ligand to a target molecule that is not endogenous to the sample. For example, an antigen endogenous to the sample is bound by a primary antibody. The primary antibody bound to the antigen is bound by a secondary antibody, e.g., an anti-immunoglobulin antibody that is covalently linked to streptavidin. The streptavidin present in the dual-specific ligand is used to capture the biotin-containing tether.

The method includes contacting the biotin-labeled tether with an avidin-containing molecule further contains a plurality of biotin molecules, thereby increasing the number of biotin molecules bound to the molecule for detection. The assembled complex is then contacted with a streptavidin-linked detectable label, e.g., an enzyme, a fluorophore, a dense particle, a microparticle, etc. for detection.

### EQUIVALENTS

Those skilled in the art will recognize, or be able to ascertain using no more than routine experimentation, many equivalents to the specific embodiments and methods described herein. Such equivalents are intended to be encompassed by the scope of the following claims.

A list of values contained within this application, e.g., the list of molecular weights for payload molecules or the number of payload molecules set forth above, are intended to include the individual value, and ranges of values bracketed by any of the listed values as upper and lower limits.

### INCORPORATION BY REFERENCE

Each reference, patent, and patent application referred to in the instant application is hereby incorporated by reference as if each reference were noted to be incorporated individually. This application is related to PCT application PCT/US2012/043366 which is incorporated herein by reference in its entirety.

1. A method of delivering a payload in a mixed payload polymer to a target molecule, the method comprising,
contacting the target molecule with a bispecific ligand comprising a first binding site and a second binding site wherein the first binding site binds specifically to a payload and the second binding site binds specifically to the target which is not the payload;
contacting the bispecific ligand with a mixed payload polymer comprising a tether molecule linked to two or more distinct payload molecules, wherein the distinct payload molecules comprise a distinct payload molecule that is the smallest payload molecule and at least one distinct payload molecule that is not the smallest payload molecule, wherein the bispecific ligand does not bind specifically to the smallest payload molecule;
wherein the payload is delivered to the target molecule, and
a) the detection sensitivity of a molecule for detection is 10⁻¹⁵ g/ml or less, and the molecule for detection is the target or is bound by the target,
b) the payload molecules attached to the tether have a molecular weight of at least 10 kDa,
c) at least 2-fold more payload is delivered to the target site using a bispecific ligand that binds to a payload molecule that is not the smallest payload molecule as compared to a bispecific ligand that binds to a payload molecule that is the smallest payload molecule, or
d) the payload molecules attached to the tether have at least 50% of the activity of a molar equivalent of payload molecules not attached to a tether.

2.-4. (canceled)

5. A method of delivering a payload in a mixed payload polymer to a nucleic acid target molecule, the method comprising,
contacting the target molecule with a bispecific ligand comprising a first binding site and a second binding site wherein the first binding site binds specifically to a payload and the second binding site binds specifically to the nucleic acid target molecule which is not the payload;
contacting the bispecific ligand with a mixed payload polymer comprising a tether molecule linked to two or more distinct payload molecules, wherein the distinct payload molecules comprise a distinct payload molecule that is the smallest payload molecule and at least one distinct payload molecule that is not the smallest payload molecule, wherein the bispecific ligand does not bind specifically to the smallest payload molecule;
wherein the payload is delivered to the nucleic acid target molecule.

6.-10. (canceled)

11. The method of claim 1, wherein the payload bound by the bispecific ligand is the largest distinct payload in the mixed payload polymer, or wherein the payload bound by the bispecific ligand is not the largest distinct payload in the mixed payload polymer.

12.-14. (canceled)

15. The method of claim 1, wherein the payload comprises a therapeutic agent, a detectable label, a nucleic acid, or an in vivo diagnostic agent.

16.-28. (canceled)

29. The method of claim 1, wherein the tether comprises a nanopolymer.

30.-34. (canceled)

35. The method of claim 1,
wherein the mixed payload polymer further comprises a biotin moiety, wherein the biotin is not a payload molecule, or
wherein the mixed payload polymer further comprises an avidin moiety selected from the group consisting of avidin, strepavidin, and neutravidin wherein the avidin moiety is not a payload molecule.

36.-52. (canceled)

53. The method of claim 1, wherein the first binding site of the bispecific ligand is comprised in a molecule selected from the group consisting of antibody, antibody fragment, affibody, T-cell receptor, nucleic acid, hapten, hormone, cytokine, receptor, receptor ligand, therapeutic agent, chelator, heavy metal, polypeptide, small molecule, hormone, cytokine, therapeutic agent, imaging agent, detectable label, antigen, receptor, biotin, streptavidin, avidin, and neutravidin.

54. The method of claim 1, wherein the second binding site of the bispecific ligand is comprised in a molecule selected from the group consisting of antibody, antibody fragment, affibody, T-cell receptor, nucleic acid, hapten, hormone, cytokine, receptor, receptor ligand, therapeutic agent, chelator, heavy metal, polypeptide, small molecule, hormone, cytokine, therapeutic agent, imaging agent, detectable label, antigen, receptor, biotin, streptavidin, avidin, and neutravidin.

55.-82. (canceled)

83. The method of claim 1, wherein the contacting comprises contacting in a subject in vivo.

84.-95. (canceled)

96. A method of detecting a target nucleic acid sequence in a sample, wherein the target nucleic acid sequence is detected without nucleic acid amplification.

97.-101. (canceled)

102. A mixed payload polymer comprising a tether molecule linked to two or more distinct payload molecules, wherein at least one of the distinct payload molecules is an active payload molecule, and the active payload molecules attached to the tether have at least 50% of the activity of a molar equivalent of active payload molecules not attached to a tether.

103.-141. (canceled)

142. A population of mixed payload polymers of claim 102, wherein each of the distinct payload molecules are each present at a molar ratio to the tethers at a ratio of at least 1:1.

143.-145. (canceled)

146. A bispecific ligand comprising a first binding site and a second binding site wherein the first binding site binds specifically to a payload molecule and the second binding site binds specifically to a target that is not the payload molecule bound by the first site.

147.-148. (canceled)

149. The bispecific ligand of claim 146, wherein the first binding site is comprised in a molecule selected from the group consisting of antibody, antibody fragment, T-cell receptor, nucleic acid, hapten, hormone, cytokine, receptor, receptor ligand, therapeutic agent, chelator, heavy metal, polypeptide, small molecule, hormone, cytokine, therapeutic agent, imaging agent, detectable label, antigen, receptor, biotin, streptavidin, avidin, and neutravidin.

150. The bispecific ligand of claim 146, wherein the second binding site is comprised in a molecule selected from the group consisting of antibody, antibody fragment, T-cell receptor, nucleic acid, hapten, hormone, cytokine, receptor, receptor ligand, therapeutic agent, chelator, heavy metal, polypeptide, small molecule, hormone, cytokine, therapeutic agent, imaging agent, detectable label, antigen, receptor, biotin, streptavidin, avidin, and neutravidin.

151.-165. (canceled)

166. A kit comprising a mixed payload polymer of claim 102 comprising a payload, and a bispecific ligand comprising a first binding site and a second binding site wherein the first binding site binds specifically to the payload and the second binding site binds specifically to a target that is not the payload.

167. (canceled)

168. A kit comprising a mixed payload polymer of claim 102 comprising a payload, a first molecule comprising a binding site that specifically binds to the payload on the mixed payload polymer, and at least one of
a) a reagent for covalently linking the first molecule comprising a binding site to a second molecule comprising a binding site;
b) a device for separating a covalently linked first molecule comprising a binding site and a second molecule comprising a binding site from a non-covalently linked first molecule comprising a binding site and a non-covalently linked second molecule comprising a binding site; and
c) a reagent for detecting a payload molecule.

169.-176. (canceled)
